(19) United States
(12) Patent Application Publication
(10) Pub. No.: US 2015/0126530 A1
(43) Pub. Date: May 7, 2015

(54) Novel Substituted Imidazopyrimidines as Gpbar1 Receptor Modulators

(71) Applicant: Torrent Pharmaceuticals Limited., Ahmedabad (IN)

(72) Inventors: Chaitanya Dutt, Gandhinagar (IN); Vijay Chauthaiwale, Gandhinagar (IN); Ramesh Chandra Gupta, Gandhinagar (IN); Sameer Ghalsasi, Gandhinagar (IN); Davinder Tuli, Gandhinagar (IN); Shailesh Deshpande, Gandhinagar (IN); Anita Chaudhari, Gandhinagar (IN); Shitalkumar Zambad, Gandhinagar (IN)

(73) Assignee: Torrent Pharmaceuticals Limited, Ahmedabad (IN)

(21) Appl. No.: 14/352,293

(22) PCT Filed: Oct. 15, 2012
(86) PCT No.: PCT/IB2012/055598

§ 371 Date: Apr. 16, 2014

(30) Foreign Application Priority Data
Oct. 21, 2011 (IN) 1352/KOL/2011

(51) Int. Cl. C07D 487/04

(52) U.S. Cl. CPC C07D 487/04
USPC 514/259.1; 544/281

(57) ABSTRACT

The present invention relates to novel substituted imidazo[1,2-a]pyrimidine compounds of formula (I), their pharmaceutically acceptable salts, and their isomers, stereoisomers, conformers, tautomers, polymorphs, hydrates and solvates. The present invention also encompasses pharmaceutically acceptable compositions of said compounds and process for preparing novel compounds. The invention further” relates to the use of the above-mentioned compounds for the preparation of medicament for use as pharmaceuticals. (Formula I).

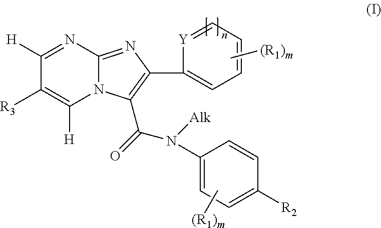

### FIELD OF THE INVENTION

The present invention relates to novel substituted imidazo[1,2-a]pyrimidine compounds of formula (I), their pharmaceutically acceptable salts, and their isomers, stereoisomers, conformers, tautomers, polymorphs, hydrates and solvates. The present invention also encompasses pharmaceutically acceptable compositions of said compounds and process for preparing the same. The invention further relates to the use of the above-mentioned compounds for the preparation of medicament for use as pharmaceuticals.

### BACKGROUND OF THE INVENTION

Diabetes is a clinical condition in which the blood glucose levels elevate because of inadequate insulin secretion to handle blood glucose and/or inadequate response to insulin (insulin resistance). Type II Diabetes (NIDDM) is a growing threat to the global health by virtue of its association with cluster of diseases that includes glucose intolerance, insulin resistance, obesity, dyslipidemia and hypertension, collectively known as metabolic syndrome. Data from global studies demonstrates that the number of people with diabetes in 2011 has reached a staggering 366 million and 4.6 million deaths are due to diabetes (International Diabetes Federation, September, 2011). Obesity, a common metabolic syndrome associated with diabetes, is second leading cause of preventable death which results from a complex interaction of genetic, behavioral and environmental factors causing an imbalance between energy intake and energy expenditure. It is well documented that patient with metabolic syndrome have higher risk for coronary heart disease and stroke (Grundy et. al. Circulation, 112: 2735, 2005).

Type II diabetes and obesity are currently treated at several levels, starting from first level therapy of diet and/or exercise alone or in combination of therapeutic agents to insulin injections.

Available therapies have proved inadequate to fulfill existing need for treatment of diabetes, obesity and associated metabolic disorders as it is evident by the figure demonstrating increased incidence and complications. Therefore, there exists need for better therapeutic approach which can treat primary conditions such as diabetes and obesity and reduces the risk of associated complications such as metabolic syndromes.

Bile acids play essential roles in the absorption of dietary lipids and cholesterol catabolism. In recent years, an important role for bile acids as signaling molecules has emerged that can activate bile acid receptors to initiate signaling pathways and regulate gene expression. Bile acids are ligands for variety of receptors including Farnesoid X receptor (FXR) and TGR5 (Gpbar1). Through activation of these receptors, bile acids can regulate their own synthesis, storage, enterohepatic circulation and also triglyceride, cholesterol, energy and glucose homeostasis.

TGR5, in the literature termed, Gpbar1, GPR131 or BG37, was identified as a G-protein coupled receptor (GPCR) responsive to bile acids (Kawamata et al, JBC 2003, 278, 9435). TGR5 is ubiquitously expressed but its expression levels vary in different tissues, with high expression in liver, intestine, brown adipose tissue and spleen. Watanabe (Nature 2006, 439, 7075) showed that the administration of bile acid to mice increases energy expenditure in brown adipose tissue, preventing obesity and insulin resistance. Bile acid binds to TGR5 and induces thyroid hormone activating enzyme type 2 idothyronine deiodinase (D2), which converts locally available thyroxine (T4) to tri-iodothyronine (T3), resulting in increased energy expenditure without leading to changes in circulating thyroid hormone levels (Trends in pharmacological sciences, 30, 11, 2009 570-580).

In addition to involvement of TGR5 in energy homeostasis, bile acid activation of membrane receptor has also been reported to promote the production of glucagon-like peptide 1 (GLP-1) in murine enteroendocrine cell lines (Katsuma et al BBRC 2005, 329, 386-390). GLP-1 stimulates insulin release in glucose dependent manner in humans Studies have also demonstrated that GLP-1 is necessary for normal glucose homeostasis. In addition, GLP-1 can exert several effects in diabetes and obesity including 1) increased glucose disposal, 2) suppression in glucose production, 3) reduced gastric emptying, and 4) reduction in food intake and weight loss.

It has been also demonstrated that activation of TGR5 prevents atherosclerotic lesion formation in Ldlr−/− mice, a commonly used mouse model of atherosclerosis, through an effect on macrophage foam cell formation (Pols et al., 2011 Cell Metabolism 14, 747-757). TGR5 is highly expressed in resting CD14+ monocytes in fractionated human leukocytes, adherent alveolar macrophage cells, and Kupffer cells in liver, indicating a potential role of TGR5 in modulating inflammation. Activation of TGR5 in Kupffer cells (Keitel et al., Biochemical and Biophysical Research Communications, 372, 1, 78-84, 2008) and THP-1 cells over expressing TGR5 (Kawamata et al., Journal of Biological Chemistry, 278, 11, 9435-9440, 2003) suppressed lipopolysaccharide- (LPS-) induced productions of cytokines, suggesting that TGR5 is a mediator in the suppression of macrophage functions by BAs.

These effects of TGR5 receptors are very relevant in metabolic syndrome as the low grade inflammation contributes to the development of metabolic syndrome. Thus, TGR5 agonist has shown potential in the intervention of metabolic syndrome and vascular and hepatic inflammatory conditions such as atherosclerosis and Non Alcoholic Fatty Liver Disease (NAFLD) (Pols et al., 2011 J Hepatol. 2011 June; 54(6):1263-72).

Experimental evidences leads to the fact that TGR 5 may play a potential role in type-2 diabetes and energy metabolism and inflammation & foam cell formation, which makes it a novel attractive target for the treatment of cardiometabolic disorders including diabetes, obesity, dyslipidemia, metabolic syndrome, atherosclerosis and non alcoholic fatty liver disease.

Recently, 3-Aryl-4-isoxazolecarboxamides have been disclosed by Evans et al, as TGR5 receptor agonists which are potentially useful therapeutics for metabolic disorders such as type II diabetes and its associated complications (JMC, 2009, vol 52, no 24, 7962-65). US 20080031968 discloses a method of treating a human for a disease or condition selected from the group consisting of hypothyroidism; hypertriglyceridemia occurring without obesity or diabetes; thyroid dysfunction; resistance to thyroid hormone; low T3 syndrome; Wilson's syndrome; depression; attention deficit disorder; insulin resistance occurring without diabetes or obesity; glucose intolerance occurring without diabetes or obesity; hypertension; infertility; cardiac insufficiency; Alzheimer's disease, Parkinson's disease; autism; and the aging process; said process comprising administering to said human a therapeutically effective amount of an agonist of the G protein coupled receptor TGR5.

WO 2004067008 discloses fused heterocyclic compounds as TGR5 receptor agonist, which is useful in treating various diseases.

International publications WO 2008067222 A1 discloses pyrrolo[1,2-a][1,4-diazepine derivatives useful as modulators of TGR5 and method for the treatment of prevention of metabolic, cardiovascular and inflammatory disease.

WO 2011057145 and WO 2011113606 discloses certain organic compounds like imidazopyridines, synthesis thereof and method of using same to treat or prevent tuberculosis in a subject or to inhibit fungal growth on plant species.

There still exists need in art to provide novel small compounds, which are TGR5 modulators.

The compounds of the present invention provide a novel substituted imidazo[1,2-a]pyrimidine compounds that binds to Gpbar1 receptors and thus useful for treatment of cardiometabolic disorders including diabetes, obesity, dyslipidemia, metabolic syndrome, atherosclerosis and non alcoholic fatty liver disease.

### SUMMARY OF THE INVENTION

In one embodiment, the present invention provides novel compounds of formula (I),

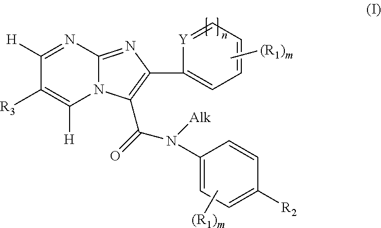

their pharmaceutically acceptable salts and their isomers, stereoisomers, conformers, tautomers, polymorphs, hydrates and solvates;
wherein,

n=0or 1;

m=0, 1, 2 or 3;

Y═CH, N or S, wherein ring containing Y group, may be optionally attached to imidazole ring through —NH, —N(alkyl), O or S;

Alk is optionally substituted straight chain alkyl, branched chain alkyl or cycloalkyl;

R₁ is selected from the group consisting of hydrogen, halo, cyano, nitro, C₁₋₈alkyl, hydroxy, —O—C₁₋₈alkyl, —CF₃, —OCF₃, —N(R₄)(CO-alkyl), —N(R₄)(SO₂-aryl), —N(R₄)(SO₂-heteroaryl), —N(R₄)(SO₂-heterocyclyl), —N(R₄)(C(O)O—R₄), —N(R₄)(C(O)O-aryl), —N(R₄)(C(O)O-heteroaryl), —N(R₄)(C(O)O-heterocyclyl), —N(R₄)C(O)N(R₄)(R₄), —N(R₄)C(O)N(R₄)(aryl), —N(R₄)C(O)N(R₄)(heteroaryl), —N(R₄)C(O)N(R₄)(heterocyclyl), —N(R₄)SO₂N(R₄)(R₄), —N(R₄)SO₂N(R₄)(aryl), —N(R₄)SO₂N(R₄)(heteroaryl), —N(R₄)SO₂N(R₄)(heterocyclyl), —OC(O)(R₄), —O(aryl), —O(heteroaryl), —O(heterocyclyl), —S(R₄), —S-aryl, —S-heteroaryl, —S-heterocyclyl, —N(R₄)(R₄), —N(R₄)(aryl), —N(R₄)(heteroaryl), —N(R₄)(heterocyclyl), —C(O)(R₄), —C(O)(aryl), —C(O)(hetero aryl), —C(O)(heterocyclyl), —C(O)N(R₄)(R₄), —C(O)N(R₄)(aryl), —C(O)N(R₄)(heteroaryl), —C(O)N(R₄)(heterocyclyl), —C(O)O—(R₄), —C(O)O-aryl, —C(O)O-heteroaryl, —C(O)O-heterocyclyl, —S(O)(aryl), —S(O)(heteroaryl), —S(O)(heterocyclyl), —SO₂(aryl), —SO₂(heteroaryl), —SO₂(heterocyclyl), —SO₂N(R₄)(R₄), —SO₂N(R₄)(aryl), —SO₂N(R₄)(heteroaryl), —SO₂N(R₄)(heterocyclyl), aryl, heteroaryl and heterocyclyl;

R₂ is selected from the group consisting of halo, cyano, nitro, C₁₋₈alkyl, hydroxy, CF₃, —OCF₃, -amino, —O(C₁₋₈alkyl), —O(aryl), —O(heteroaryl), —O(heterocyclyl), —S(R₄), —S-aryl, —S-heteroaryl, —S-heterocyclyl, —C(O)O—(R₄), —C(O)O-aryl, —C(O)O-heteroaryl, —C(O)O— heterocyclyl, —SO₂N(R₄)(R₄), —SO₂N(R₄)(aryl), —SO₂N(R₄)(heteroaryl), —SO₂N(R₄)(heterocyclyl), aryl, heteroaryl and heterocyclyl; or

R₁ & R₂ when present on adjacent carbon atom may join together to form cycloalkenyl, aryl, heteroaryl or heterocyclyl ring;

R₃ is selected from the group consisting of hydrogen, cyano, nitro, hydroxy, —O(C₁₋₈ alkyl), —OCF₃, —N(R₄)(CO—R₄), —N(R₄)(CO-aryl), —N(R₄)(CO-heteroaryl), —N(R₄)(SO₂—R₄), —N(R₄)(SO₂—CF₃), —N(R₄)(SO₂-aryl), —N(R₄)(SO₂-heteroaryl), —N(R₄)(SO₂-heterocyclyl), —N(R₄)(C(O)O—R₄), —N(R₄)(C(O)O-aryl), —N(R₄)(C(O)O-heteroaryl), —N(R₄)(C(O)O— heterocyclyl), —N(R₄)C(O)N(R₄)(R₄), —N(R₄)C(O)N(R₄)(aryl), —N(R₄)C(O)N(R₄)(heteroaryl), —N(R₄)C(O)N(R₄)(heterocyclyl), —O(aryl), —O(heteroaryl), —O(heterocyclyl), —S(R₄), —S-aryl, —S-heteroaryl, —S-heterocyclyl, —N(R₄)(R₄), —N(R₄)(aryl), —N(R₄)(heteroaryl), —N(R₄)(heterocyclyl), —C(O)(R₄), —C(O)(aryl), —C(O)(heteroaryl), —C(O)(heterocyclyl), —C(O)N(R₄)(R₄), —C(O)N(R₄)(aryl), —C(O)N(R₄)(heteroaryl), —C(O)N(R₄)(heterocyclyl), —C(O)O—(R₄), —C(O)O-aryl, —C(O)O-heteroaryl, —C(O)O-heterocyclyl, S(O)—(C₁₋₈alkyl), —S(O)(aryl), —S(O)(heteroaryl), —S(O)(heterocyclyl), —SO₂ (C₁₋₈alkyl), —SO₂(aryl), —SO₂(heteroaryl), —SO₂(heterocyclyl), —SO₂N(R₄)(R₄), —SO₂N(R₄)(aryl), SO₂N(R₄)(heteroaryl), —SO₂N(R₄)(heterocyclyl) and —SO₂N(R₄)(cycloalkyl); and

R₄ is hydrogen or —C₁₋₈alkyl.

In another embodiment, the present invention provides novel compounds of formula (I),

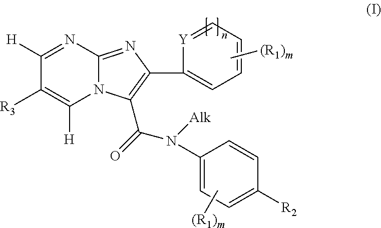

their pharmaceutically acceptable salts and their isomers, stereoisomers, conformers, tautomers, polymorphs, hydrates and solvates;
wherein,

n=0 or 1;

m=0, 1, 2 or 3;

Y═CH, N or S, wherein ring containing Y group, may be optionally attached to imidazole ring through —NH, —N(alkyl), O or S;

Alk is optionally substituted straight chain alkyl, branched chain alkyl or cycloalkyl;

R₁ is selected from the group consisting of hydrogen, halo, cyano, C₁₋₈alkyl, hydroxy, —CF₃, —OCF₃, —N(R₄)(SO₂-aryl), —N(R₄)(SO₂-heteroaryl), —N(R₄)(SO₂-heterocyclyl), —N(R₄)(C(O)O—R₄), —N(R₄)(C(O)O-aryl), —N(R₄)(C(O)O-heteroaryl), —N(R₄)(C(O)O— heterocyclyl), —N(R₄)C(O)N(R₄)(R₄), —N(R₄)SO₂N(R₄)(aryl), _—O(aryl), —S(R₄), —N(R₄)(R₄), —N(R₄)(aryl), —C(O)(heterocyclyl), —C(O)N(R₄)(R₄), —C(O)N(R₄)(aryl), —SO₂(aryl), —SO₂N(R₄)(R₄), —SO₂N(R₄)(aryl), aryl, heteroaryl and heterocyclyl;

R₂ is selected from the group consisting of halo, cyano, C₁₋₈alkyl, hydroxy, CF₃, —OCF₃, -amino, —O(C₁₋₈alkyl), —O(aryl), —S-aryl, —C(O)O—(R₄), —SO₂N(R₄)(R₄), —SO₂N(R₄)(aryl), aryl, heteroaryl and heterocyclyl; or

R₁ & R₂ when present on adjacent carbon atom may join together to form cycloalkenyl, aryl, heteroaryl or heterocyclyl ring;

R₃ is selected from the group consisting of hydrogen, cyano, nitro, hydroxy, —O(C₁₋₈alkyl), —N(R₄)SO₂(aryl), —N(R₄)(C(O)O—R₄), —N(R₄)C(O)N(R₄)(R₄),—N(R₄)C(O)N(R₄)(aryl), —N(R₄)(R₄), —C(O)(heterocyclyl),—C(O)O—(R₄), —SO₂(aryl) and —SO₂N(R₄)(aryl); and

R₄ is hydrogen or —C₁₋₈alkyl.

In another embodiment, the present invention provides novel compounds of formula (I),

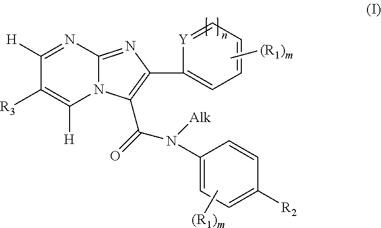

their pharmaceutically acceptable salts and their isomers, stereoisomers, conformers, tautomers, polymorphs, hydrates and solvates;
wherein,

n=0 or 1;

m=0, 1 or 2;

Y═CH, N or S, wherein ring containing Y group, may be optionally attached to imidazole ring through —NH or O;

Alk is optionally substituted straight chain alkyl, branched chain alkyl or cycloalkyl;

R₁ is selected from the group consisting of hydrogen, halo, cyano, C₁₋₈alkyl, hydroxy, —CF₃, —OCF₃, —N(R₄)(R₄), —SO₂N(R₄)(R₄), aryl, heteroaryl and heterocyclyl;

R₂ is selected from the group consisting of halo, C₁₋₈alkyl, hydroxy, —O(C₁₋₈alkyl), —C(O)O—(R₄), —SO₂N(R₄)(R₄) and heterocyclyl; or

R₁ & R₂ when present on adjacent carbon atom may join together to form cycloalkenyl, ring;

R₃ is selected from the group consisting of hydrogen, hydroxy, —N(R₄)(C(O)O—R₄), N(R₄)C(O)N(R₄)(R₄), —N(R₄)(R₄), —C(O)(heterocyclyl), C(O)O—(R₄) and SO₂(aryl); and

R₄ is hydrogen or —C₁₋₈alkyl.

In another embodiment, the present invention pertains to compounds as above, however only including pharmaceutically acceptable salts thereof.

In another embodiment, the present invention provides a compound N-(4-chlorophenyl)-2-(4-fluorophenyl)-N-methylimidazo[1,2-a]pyrimidine-3-carboxamide or pharmaceutically acceptable salts and their isomers, stereoisomers, conformers, tautomers, polymorphs, hydrates and solvates thereof.

In another embodiment, the present invention provides a compound N-(4-chlorophenyl)-2-(4-fluorophenoxy)-N-methylimidazo[1,2-a]pyrimidine-3-carboxamide or pharmaceutically acceptable salts and their isomers, stereoisomers, conformers, tautomers, polymorphs, hydrates and solvates thereof.

In another embodiment, the present invention includes synthetic intermediates that are useful in preparing the compounds of formula (I) and process for preparing such intermediates.

Another embodiment of the present invention is a method for preparation of compounds of formula (I) as herein described in Scheme I & II.

Another embodiment of the present invention is a pharmaceutical composition comprising compounds of formula (I), optionally in admixture with a pharmaceutically acceptable adjuvant, diluent or carrier.

Another embodiment of the present invention is a method for treating cardiometabolic disorders including diabetes, obesity, dyslipidemia, metabolic syndrome, atherosclerosis and non alcoholic fatty liver disease by administering a therapeutically effective amount of compounds of formula (I) to a mammal in need thereof.

Another embodiment of the present invention is the use of compounds of formula (I) for the preparation of a medicament for treating cardiometabolic disorders including diabetes, obesity, dyslipidemia, metabolic syndrome, atherosclerosis and non alcoholic fatty liver disease.

Another embodiment of the present invention is a pharmaceutical composition comprising N-(4-chlorophenyl)-2-(4-fluorophenyl)-N-methylimidazo[1,2-a]pyrimidine-3-carboxamide or pharmaceutically acceptable salts and their isomers, stereoisomers, conformers, tautomers, polymorphs, hydrates and solvates thereof, optionally in admixture with a pharmaceutically acceptable adjuvant, diluent or carrier.

Another embodiment of the present invention is a pharmaceutical composition comprising N-(4-chlorophenyl)-2-(4-fluorophenoxy)-N-methylimidazo[1,2-a]pyrimidine-3-carboxamide or pharmaceutically acceptable salts and their isomers, stereoisomers, conformers, tautomers, polymorphs, hydrates and solvates thereof, optionally in admixture with a pharmaceutically acceptable adjuvant, diluent or carrier.

Another embodiment of the present invention is a method of treating cardiometabolic disorders including diabetes, obesity, dyslipidemia, metabolic syndrome, atherosclerosis and non alcoholic fatty liver disease by administering a therapeutically effective amount of a N-(4-chlorophenyl)-2-(4-fluorophenyl)-N-methylimidazo[1,2-a]pyrimidine-3-carboxamide or pharmaceutically acceptable salts and their isomers, stereoisomers, conformers, tautomers, polymorphs, hydrates and solvates thereof to a mammal in need thereof.

Another embodiment of the present invention is a method of treating cardiometabolic disorders including diabetes, obesity, dyslipidemia, metabolic syndrome, atherosclerosis and non alcoholic fatty liver disease by administering a therapeutically effective amount of a N-(4-chlorophenyl)-2-(4-fluorophenoxy)-N-methylimidazo[1,2-a]pyrimidine-3-carboxamide or pharmaceutically acceptable salts and their isomers, stereoisomers, conformers, tautomers, polymorphs, hydrates and solvates thereof to a mammal in need thereof.

Another embodiment of the present invention is the use of a N-(4-chlorophenyl)-2-(4-fluorophenyl)-N-methylimidazo[1,2-a]pyrimidine-3-carboxamide or pharmaceutically acceptable salts and their isomers, stereoisomers, conformers, tautomers, polymorphs, hydrates and solvates thereof for the preparation of a medicament for treating cardiometabolic disorders including diabetes, obesity, dyslipidemia, metabolic syndrome, atherosclerosis and non alcoholic fatty liver disease.

Another embodiment of the present invention is the use of a N-(4-chlorophenyl)-2-(4-fluorophenoxy)-N-methylimidazo[1,2-a]pyrimidine-3-carboxamide or pharmaceutically acceptable salts and their isomers, stereoisomers, conformers, tautomers, polymorphs, hydrates and solvates thereof for the preparation of a medicament for treating cardiometabolic disorders including diabetes, obesity, dyslipidemia, metabolic syndrome, atherosclerosis and non alcoholic fatty liver disease.

Another embodiment of the present invention provides the use of compound of formula (I) for the preparation of salts, polymorphs, hydrates and solvates of compound of formula (I).

### DETAILED DESCRIPTION OF THE INVENTION

In one embodiment, the present invention provides novel compounds of formula (I),

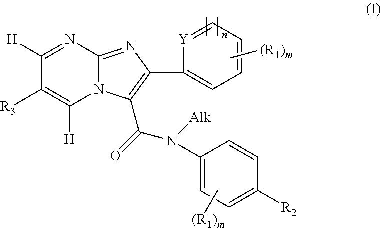

their pharmaceutically acceptable salts and their isomers, stereoisomers, conformers, tautomers, polymorphs, hydrates, and solvates, wherein R₁, R₂, R₃, R₄, Y, m and n are as defined herein above.

A family of specific compounds of particular interest within the above formula (I) consists of compound and pharmaceutically acceptable salts thereof as follows:

| Comp. No. | Chemical Name |
|---|---|
| 1 | N-(4-chlorophenyl)-N-methyl-2-phenylimidazo[1,2-a]pyrimidine-3-carboxamide |
| 2 | N-(3-chlorophenyl)-N-methyl-2-phenylimidazo[1,2-a]pyrimidine-3-carboxamide |
| 3 | (4-{methyl[(2-phenylimidazo[1,2-a]pyrimidin-3-yl)carbonyl] amino}phenyl) |
| | acetic acid |
| 4 | 2-(4-chlorophenyl)-N-(2,3-dihydro-1H-inden-5-yl)-N-methylimidazo[1,2-a] |
| | pyrimidine-3-carboxamide |
| 5 | N,2-bis(4-chlorophenyl)-N-methylimidazo[1,2-a]pyrimidine-3-carboxamide |
| 6 | N-(2,3-dihydro-1H-inden-5-yl)-2-(4-fluorophenyl)-N-methylimidazo[1,2-a] |
| | pyrimidine-3-carboxamide |
| 7 | N-(4-chlorophenyl)-2-(4-fluorophenyl)-N-methylimidazo[1,2-a]pyrimidine-3- |
| | carboxamide |
| 8 | 2-(biphenyl-4-yl)-N-(4-chlorophenyl)-N-methylimidazo[1,2-a]pyrimidine-3- |
| | carboxamide |
| 9 | 2-(biphenyl-4-yl)-N-(2,3-dihydro-1H-inden-5-yl)-N-methylimidazo[1,2-a] |
| | pyrimidine-3-carboxamide |
| 10 | N-(4-chlorophenyl)-2-(3,4-dimethoxyphenyl)-N-methylimidazo[1,2-a] |
| | pyrimidine-3-carboxamide |
| 11 | N-(4-chlorophenyl)-N-methyl-2-(pyridin-2-yl)imidazo[1,2-a]pyrimidine-3- |
| | carboxamide |
| 12 | N-(4-chlorophenyl)-N-methyl-2,6-diphenylimidazo[1,2-a]pyrimidine-3- |
| | carboxamide |
| 13 | N-(2,3-dihydro-1H-inden-5-yl)-2-(3,4-dimethoxyphenyl)-N- |
| | methylimidazo[1,2-a]pyrimidine-3-carboxamide |
| 14 | N-(4-chlorophenyl)-2-(3,4-dichlorophenyl)-N-methylimidazo[1,2-a] |
| | pyrimidine-3-carboxamide |
| 15 | N-(2,3-dihydro-1H-inden-5-yl)-2-(3,4-dihydroxyphenyl)-N- |
| | methylimidazo[1,2-a]pyrimidine-3-carboxamide |
| 16 | N-(4-chlorophenyl)-2-(4-methoxyphenyl)-N-methylimidazo[1,2-a]pyrimidine- |
| | 3-carboxamide |
| 17 | N-(2,3-dihydro-1H-inden-5-yl)-N-methyl-2-(pyridin-2-yl)imidazo[1,2-a] |
| | pyrimidine-3-carboxamide |
| 18 | 2-(4-chlorophenyl)-N-(4-fluorophenyl)-N-methylimidazo[1,2-a]pyrimidine-3- |
| | carboxamide |
| 19 | 2-(4-chlorophenyl)-N-(3,4-dimethoxyphenyl)-N-methylimidazo[1,2-a] |
| | pyrimidine-3-carboxamide |
| 20 | N-(4-chlorophenyl)-N-methyl-2-(pyridin-3-yl)imidazo[1,2-a]pyrimidine-3- |
| | carboxamide |
| 21 | 2-(2,4-dichlorophenyl)-N-(4-fluorophenyl)-N-methylimidazo[1,2-a] |
| | pyrimidine-3-carboxamide |
| 22 | N,2-bis(4-fluorophenyl)-N-methylimidazo[1,2-a]pyrimidine-3-carboxamide |
| 23 | N-(4-chlorophenyl)-N-methyl-2-[2-(trifluoromethyl)phenyl]imidazo[1,2-a] |
| | pyrimidine-3-carboxamide |
| 24 | 2-(4-chlorophenyl)-N-[4-(dimethylsulfamoyl)phenyl]-N-methylimidazo[1,2-a] |
| | pyrimidine-3-carboxamide |
| 25 | 2-(4-chlorophenyl)-N-(2,4-dimethoxyphenyl)-N-methylimidazo[1,2-a] |
| | pyrimidine-3-carboxamide |
| 26 | N-(4-fluorophenyl)-N-methyl-2-phenylimidazo[1,2-a]pyrimidine-3-carboxamide |
| 27 | 2-(4-chlorophenyl)-N-(3,4-dihydroxyphenyl)-N-methylimidazo[1,2-a] |
| | pyrimidine-3-carboxamide |
| 28 | 2-(1,3-benzodioxol-5-yl)-N-(4-fluorophenyl)-N-methylimidazo[1,2-a] |
| | pyrimidine-3-carboxamide |
| 29 | 2-(1,3-benzodioxol-5-yl)-N-(4-chlorophenyl)-N-methylimidazo[1,2-a] |
| | pyrimidine-3-carboxamide |
| 30 | N-(4-chlorophenyl)-2-(4-hydroxyphenyl)-N-methylimidazo[1,2-a]pyrimidine- |
| | 3-carboxamide |
| 31 | N-(4-chlorophenyl)-N-methyl-2-[3-(trifluoromethyl)phenyl]imidazo[1,2-a] |
| | pyrimidine-3-carboxamide |
| 32 | N-(4-chlorophenyl)-2-(4-fluorophenyl)imidazo[1,2-a]pyrimidine-3-carboxamide |
| 33 | N-(4-fluorophenyl)-N-methyl-2-[3-(trifluoromethyl)phenyl]imidazo[1,2-a] |
| | pyrimidine-3-carboxamide |
| 34 | N-(2,4-dimethoxyphenyl)-2-(4-fluorophenyl)imidazo[1,2-a]pyrimidine-3- |
| | carboxamide |
| 35 | 2-(3-cyanophenyl)-N-(4-fluorophenyl)-N-methylimidazo[1,2-a]pyrimidine-3- |
| | carboxamide; |
| 36 | N-(4-chlorophenyl)-2-(3-cyanophenyl)-N-methylimidazo[1,2-a]pyrimidine-3- |
| | carboxamide |
| 37 | N-(4-chlorophenyl)-2-[4-(dimethylsulfamoyl)phenyl]-N-methylimidazo[1,2-a] |
| | pyrimidine-3-carboxamide; |
| 38 | 2-[4-(dimethylsulfamoyl)phenyl]-N-(4-fluorophenyl)-N-methylimidazo[1,2-a] |
| | pyrimidine-3-carboxamide |
| 39 | N-(2,4-difluorophenyl)-2-(4-fluorophenyl)imidazo[1,2-a]pyrimidine-3-carboxamide |
| 40 | N-(2,4-difluorophenyl)-2-(4-fluorophenyl)-N-methylimidazo[1,2-a]pyrimidine- |
| | 3-carboxamide |
| 41 | 2-(3-fluoro-4-methoxyphenyl)-N-(4-fluorophenyl)-N-methylimidazo[1,2-a] |
| | pyrimidine-3-carboxamide |
| 42 | 2-(3-fluoro-4-hydroxyphenyl)-N-(4-fluorophenyl)-N-methylimidazo[1,2-a] |
| | pyrimidine-3-carboxamide |
| 43 | N-(2,4-difluorophenyl)-2-(3-fluoro-4-methoxyphenyl)-N-methylimidazo[1,2-a] |
| | pyrimidine-3-carboxamide |
| 44 | N-(4-chlorophenyl)-2-(3-fluoro-4-methoxyphenyl)-N-methylimidazo[1,2-a] |
| | pyrimidine-3-carboxamide |
| 45 | N-(3-chloro-4-fluorophenyl)-2-(3-fluoro-4-methoxyphenyl)-N-methylimidazo[1,2-a] |
| | pyrimidine-3-carboxamide |
| 46 | N-(2,4-difluorophenyl)-2-(4-fluorophenoxy)-N-methylimidazo[1,2-a] |
| | pyrimidine-3-carboxamide |
| 47 | N-(4-chlorophenyl)-N-methyl-2-phenoxyimidazo[1,2-a]pyrimidine-3-carboxamide |
| 48 | 2-(4-chlorophenoxy)-N-(4-chlorophenyl)-N-methylimidazo[1,2-a]pyrimidine- |
| | 3-carboxamide |
| 49 | N-(4-chlorophenyl)-2-(4-methoxyphenoxy)-N-methylimidazo[1,2-a] |
| | pyrimidine-3-carboxamide |
| 50 | N-(4-chlorophenyl)-2-(4-fluorophenoxy)-N-methylimidazo[1,2-a]pyrimidine- |
| | 3-carboxamide |
| 51 | 2-(4-chlorophenoxy)-N-(4-fluorophenyl)-N-methylimidazo[1,2-a]pyrimidine- |
| | 3-carboxamide |
| 52 | 2-(4-fluorophenoxy)-N-(4-methoxyphenyl)-N-methylimidazo[1,2-a] |
| | pyrimidine-3-carboxamide |
| 53 | N-(4-chlorophenyl)-2-(3-fluoro-4-hydroxyphenyl)-N-methylimidazo[1,2-a] |
| | pyrimidine-3-carboxamide |
| 54 | N-(4-chlorophenyl)-2-(4-cyanophenyl-N-methylimidazo[1,2-a]pyrimidine-3- |
| | carboxamide |
| 55 | 2-(4-cyanophenyl)-N-(4-fluorophenyl)-N-methylimidazo[1,2-a]pyrimidine-3- |
| | carboxamide |
| 56 | N-(3-chloro-4-fluorophenyl)-2-(4-cyanophenyl)-N-methylimidazo[1,2-a] |
| | pyrimidine-3-carboxamide |
| 57 | N-(4-chlorophenyl)-N-(4-fluorobenzyl)-2-(4-fluorophenyl)imidazo[1,2-a] |
| | pyrimidine-3-carboxamide |
| 58 | 2-(2,6-dichlorophenoxy)-N-(2,4-difluorophenyl)-N-methylimidazo[1,2-a] |
| | pyrimidine-3-carboxamide |
| 59 | 2-(2,6-dichlorophenoxy)-N-(4-fluorophenyl)-N-methylimidazo[1,2-a] |
| | pyrimidine-3-carboxamide |
| 60 | N-(4-chlorophenyl)-2-(2,4-difluorophenoxy)-N-methylimidazo[1,2-a] |
| | pyrimidine-3-carboxamide |
| 61 | 2-(5-chlorothiophen-2-yl)-N-(4-fluorophenyl)-N-methylimidazo[1,2-a] |
| | pyrimidine-3-carboxamide |
| 62 | N-(4-chlorophenyl)-2-(5-chlorothiophen-2-yl)-N-methylimidazo[1,2-a] |
| | pyrimidine-3-carboxamide |
| 63 | N-(4-chlorophenyl)-N-methyl-2-[4-(trifluoromethyl)phenyl]imidazo[1,2-a] |
| | pyrimidine-3-carboxamide |
| 64 | N-(4-fluorophenyl)-N-methyl-2-[4-(trifluoromethyl)phenyl]imidazo[1,2-a] |
| | pyrimidine-3-carboxamide |
| 65 | N-(4-chlorophenyl)-2-(4-fluorophenyl)-N-(propan-2-yl)imidazo[1,2-a] |
| | pyrimidine-3-carboxamide |
| 66 | N-(4-chlorophenyl)-2-(4-fluorophenoxy)imidazo[1,2-a]pyrimidine-3- |
| | carboxamide |
| 67 | N-(4-fluorophenyl)-2-[3-(trifluoromethyl)phenyl]imidazo[1,2-a]pyrimidine-3- |
| | carboxamide |
| 68 | N-cyclopropyl-N,2-bis(4-fluorophenyl)imidazo[1,2-a]pyrimidine-3-carboxamide |
| 69 | N-cyclopropyl-N-(4-fluorophenyl)-2-[3-(trifluoromethyl)phenyl]imidazo[1,2-a] |
| | pyrimidine-3-carboxamide |
| 70 | N-(4-chlorophenyl)-N-cyclopropyl-2-[3-(trifluoromethyl)phenyl]imidazo[1,2-a] |
| | pyrimidine-3-carboxamide |
| 71 | methyl {2-(4-fluorophenyl)-3-[(4-fluorophenyl)(methyl)carbamoyl] |
| | imidazo[1,2-a]pyrimidin-6-yl}carbamate |
| 72 | methyl {3-[(4-chlorophenyl)(methyl)carbamoyl]-2-(4-fluorophenyl) |
| | imidazo[1,2-a]pyrimidin-6-yl}carbamate |
| 73 | 6-(carbamoylamino)-N-(4-chlorophenyl)-2-(4-fluorophenyl)-N-methyl |
| | imidazo[1,2-a]pyrimidine-3-carboxamide |
| 74 | 6-(carbamoylamino)-N,2-bis(4-fluorophenyl)-N-methylimidazo[1,2-a] |
| | pyrimidine-3-carboxamide |
| 75 | 6-(carbamoylamino)-2-(4-fluorophenyl)-N-(4-methoxyphenyl)-N-methylimidazo |
| | [1,2-a]pyrimidine-3-carboxamide |
| 76 | N-(4-chlorophenyl)-2-[3-(dimethylamino)phenyl]-N-methylimidazo[1,2-a] |
| | pyrimidine-3-carboxamide |
| 77 | 2-[3-(dimethylamino)phenyl)-N-(4-fluorophenyl)-N-methylimidazo[1,2-a] |
| | pyrimidine-3-carboxamide |
| 78 | N-(4-chlorophenyl)-2-(5-fluoro-2-hydroxyphenyl)-N-methylimidazo[1,2-a] |
| | pyrimidine-3-carboxamide |
| 79 | 2-(5-fluoro-2-hydroxyphenyl)-N-(4-fluorophenyl)-N-methylimidazo[1,2-a] |
| | pyrimidine-3-carboxamide |
| 80 | 2-(5-fluoro-2-hydroxyphenyl)-N-(4-methoxyphenyl)-N-methylimidazo[1,2-a] |
| | pyrimidine-3-carboxamide |
| 81 | N-(4-chlorophenyl)-N-methyl-2-[4-(propan-2-yl)phenyl]imidazo[1,2-a] |
| | pyrimidine-3-carboxamide |
| 82 | N-(4-fluorophenyl)-N-methyl-2-[4-(propan-2-yl)phenyl]imidazo[1,2-a] |
| | pyrimidine-3-carboxamide |
| 83 | N-(4-chlorophenyl)-2-(2,6-dihydroxyphenyl)-N-methylimidazo[1,2-a] |
| | pyrimidine-3-carboxamide |
| 84 | 2-(2,6-dihydroxyphenyl)-N-(4-fluorophenyl)-N-methylimidazo[1,2-a] |
| | pyrimidine-3-carboxamide |
| 85 | N-(4-chlorophenyl)-2-(2-hydroxy-6-methoxyphenyl)-N-methylimidazo[1,2-a] |
| | pyrimidine-3-carboxamide |
| 86 | N-(4-fluorophenyl)-2-(2-hydroxy-6-methoxyphenyl)-N-methylimidazo[1,2-a] |
| | pyrimidine-3-carboxamide |
| 87 | N-(4-chlorophenyl)-N-methyl-2-(thiophen-2-yl)imidazo[1,2-a]pyrimidine-3- |
| | carboxamide |
| 88 | N-(4-fluorophenyl)-N-methyl-2-(thiophen-2-yl)imidazo[1,2-a]pyrimidine-3- |
| | carboxamide |
| 89 | N-(4-chlorophenyl)-2-(5-fluoro-2-hydroxyphenyl)-N-methylimidazo[1,2-a] |
| | pyrimidine-3-carboxamide |
| 90 | 2-(5-fluoro-2-hydroxyphenyl)-N-(4-fluorophenyl)-N-methylimidazo[1,2-a] |
| | pyrimidine-3-carboxamide |
| 91 | N-(4-chlorophenyl)-2-(furan-2-yl)-N-methylimidazo[1,2-a]pyrimidine-3- |
| | carboxamide |
| 92 | N-(4-fluorophenyl)-2-(furan-2-yl)-N-methylimidazo[1,2-a]pyrimidine-3- |
| | carboxamide |
| 93 | N-(4-chlorophenyl)-2-(2,3-dihydro-1,4-benzodioxin-5-yl)-N-methylimidazo |
| | [1,2-a]pyrimidine-3-carboxamide |
| 94 | 2-(2,3-dihydro-1,4-benzodioxin-5-yl)-N-(4-fluorophenyl)-N-methylimidazo |
| | [1,2-a]pyrimidine-3-carboxamide |
| 95 | N-(4-chlorophenyl)-2-(2-fluoro-4-methoxyphenyl)-N-methylimidazo[1,2-a] |
| | pyrimidine-3-carboxamide |
| 96 | 2-(2-fluoro-4-methoxyphenyl)-N-(4-fluorophenyl)-N-methylimidazo[1,2-a] |
| | pyrimidine-3-carboxamide |
| 97 | N-(4-chlorophenyl)-N-methyl-2-[3-(trifluoromethoxy)phenyl]imidazo[1,2-a] |
| | pyrimidine-3-carboxamide |
| 98 | N-(4-fluorophenyl)-N-methyl-2-[3-(trifluoromethoxy)phenyl]imidazo[1,2-a] |
| | pyrimidine-3-carboxamide |
| 99 | N-(4-chlorophenyl)-N-methyl-2-[4-(methylsulfonyl)phenyl]imidazo[1,2-a] |
| | pyrimidine-3-carboxamide |
| 100 | N-(4-fluorophenyl)-N-methyl-2-[4-(methylsulfonyl)phenyl]imidazo[1,2-a] |
| | pyrimidine-3-carboxamide |
| 101 | N-(4-chlorophenyl)-2-[(4-methoxyphenyl)amino]-N-methylimidazo[1,2-a] |
| | pyrimidine-3-carboxamide |
| 102 | N-(4-fluorophenyl)-2-[(4-methoxyphenyl)amino]-N-methylimidazo[1,2-a] |
| | pyrimidine-3-carboxamide |
| 103 | N-(4-chlorophenyl)-2-[(4-chlorophenyl)amino]-N-methylimidazo[1,2-a] |
| | pyrimidine-3-carboxamide |
| 104 | 2-[(4-chlorophenyl)amino]-N-(4-fluorophenyl)-N-methylimidazo[1,2-a] |
| | pyrimidine-3-carboxamide |
| 105 | N-(4-chlorophenyl)-2-[(4-fluorophenyl)amino]-N-methylimidazo[1,2-a] |
| | pyrimidine-3-carboxamide |
| 106 | N-(4-fluorophenyl)-2-[(4-fluorophenyl)amino]-N-methylimidazo[1,2-a] |
| | pyrimidine-3-carboxamide |
| 107 | N-(4-chlorophenyl)-2-[(4-methoxyphenyl)amino]-N-methylimidazo[1,2-a] |
| | pyrimidine-3-carboxamide |
| 108 | N-(4-fluorophenyl)-2-[(4-methoxyphenyl)amino]-N-methylimidazo[1,2-a] |
| | pyrimidine-3-carboxamide |
| 109 | N-(4-chlorophenyl)-2-[(3-fluoro-4-hydroxyphenyl)amino]-N-methylimidazo |
| | [1,2-a]pyrimidine-3-carboxamide |
| 110 | N-(4-chlorophenyl)-N-methyl-2-{[2-(trifluoromethyl)phenyl]amino}imidazo |
| | [1,2-a]pyrimidine-3-carboxamide |
| 111 | N-(4-fluorophenyl)-N-methyl-2-{[2-(trifluoromethyl)phenyl]amino}imidazo |
| | [1,2-a]pyrimidine-3-carboxamide |
| 112 | N-(4-chlorophenyl)-N-methyl-2-{[3-(trifluoromethyl)phenyl]amino}imidazo |
| | [1,2-a]pyrimidine-3-carboxamide |
| 113 | N-(4-fluorophenyl)-N-methyl-2-{[3-(trifluoromethyl)phenyl]amino}imidazo |
| | [1,2-a]pyrimidine-3-carboxamide |
| 114 | N-(4-chlorophenyl)-6-(dimethylamino)-2-(4-fluorophenyl)-N-methylimidazo |
| | [1,2-a]pyrimidine-3-carboxamide |
| 115 | N-(4-chlorophenyl)-2-[(4-fluorophenyl)amino]-N-methylimidazo[1,2-a] |
| | pyrimidine-3-carboxamide |
| 116 | methyl {3-[(4-chlorophenyl)(methyl)carbamoyl]-2-(4-fluorophenyl)imidazo |
| | [1,2-a]pyrimidin-6-yl}methylcarbamate |
| 117 | N-(4-chlorophenyl)-2-(4-fluorophenyl)-5,7-dihydroxy-N-methylimidazo[1,2-a] |
| | pyrimidine-3-carboxamide |
| 118 | N-(4-chlorophenyl)-2-(4-fluorophenyl)-N,7-dimethylimidazo[1,2-a] |
| | pyrimidine-3-carboxamide |
| 119 | N-(4-chlorophenyl)-2-(4-fluorophenyl)-N-methyl-6-[methyl(propan-2- |
| | ylcarbamoyl)amino]imidazo[1,2-a]pyrimidine-3-carboxamide |
| 120 | N-(4-chlorophenyl)-2-(4-fluorophenyl)-7-hydroxy-N-methylimidazo[1,2-a] |
| | pyrimidine-3-carboxamide |
| 121 | N-(4-chlorophenyl)-2-(4-fluorophenyl)-7-hydroxyimidazo[1,2-a]pyrimidine-3- |
| | carboxamide |
| 122 | N-(4-chlorophenyl)-2-(4-fluorophenyl)-5-hydroxy-N-methylimidazo[1,2-a] |
| | pyrimidine-3-carboxamide |
| 123 | N-(4-chlorophenyl)-2-(4-fluorophenyl)-5-hydroxyimidazo[1,2-a]pyrimidine-3- |
| | carboxamide |
| 124 | N-(4-chlorophenyl)-2-(4-fluorophenyl)-7-hydroxy-N-(hydroxymethyl)imidazo |
| | [1,2-a]pyrimidine-3-carboxamide |
| 125 | N-(4-chlorophenyl)-2-(4-fluorophenyl)-N-(hydroxymethyl)imidazo[1,2-a] |
| | pyrimidine-3-carboxamide |
| 126 | N-(4-chlorophenyl)-2-(4-fluoro-2-hydroxyphenyl)-N-methylimidazo[1,2-a] |
| | pyrimidine-3-carboxamide |
| 127 | N-(4-chloro-2-hydroxyphenyl)-2-(4-fluorophenyl)-N-methylimidazo[1,2-a] |
| | pyrimidine-3-carboxamide |
| 128 | 3-[(4-chlorophenyl)(methyl)carbamoyl]-2-(4-fluorophenyl)imidazo[1,2-a] |
| | pyrimidine-6-carboxylic acid |
| 129 | N-(4-chlorophenyl)-2-(4-fluorophenyl)-N-methyl-6-(phenylsulfonyl)imidazo |
| | [1,2-a]pyrimidine-3-carboxamide |
| 130 | N-(4-chlorophenyl)-2-(4-fluorophenyl)-N-methyl-6-(pyrrolidin-1-ylcarbonyl) |
| | imidazo[1,2-a]pyrimidine-3-carboxamide |
| 131 | N-(4-fluorophenyl)-N-methyl-2-(morpholin-4-yl)imidazo[1,2-a]pyrimidine-3- |
| | carboxamide |
| 132 | N-(4-chlorophenyl)-2-(3-fluorophenoxy)-N-methylimidazo[1,2-a]pyrimidine- |
| | 3-carboxamide |
| 133 | 2-(4-fluorophenyl)-N-(4-methoxyphenyl)-N-methylimidazo[1,2-a]pyrimidine- |
| | 3-carboxamide |
| 134 | ethyl 4-[{[2-(4-fluorophenyl)imidazo[1,2-a]pyrimidin-3- |
| | yl]carbonyl}(methyl)amino]benzoate |
| 135 | N-(4-chlorophenyl)-2-(4-fluoro-2-methoxyphenyl)-N-methylimidazo[1,2-a] |
| | pyrimidine-3-carboxamide |
| 136 | N-(4-chlorophenyl)-N-cyclopropyl-2-(4-fluorophenyl)imidazo[1,2-a] |
| | pyrimidine-3-carboxamide |
| 137 | N-(4-chlorophenyl)-2-(4-fluorophenyl)-N-methylimidazo[1,2-a]pyrimidine-3- |
| | carboxamide hydrochloride (1:1) |
| 138 | N-(4-chlorophenyl)-2-(4-fluorophenyl)-N-methylimidazo[1,2-a]pyrimidine-3- |
| | carboxamide sulfate (1:1) |
| 139 | N-(4-chlorophenyl)-2-(4-fluorophenyl)-N-methylimidazo[1,2-a]pyrimidine-3- |
| | carboxamide 4-methylbenzenesulfonate (1:1) |
| 140 | N-(4-chlorophenyl)-2-(4-fluorophenyl)-N-methylimidazo[1,2-a]pyrimidine-3- |
| | carboxamide methanesulfonate (1:1) |
| 141 | 2-(4-fluorophenyl)-N-methylimidazo[1,2-a]pyrimidine-3-carboxamide |
| 142 | N-(4-cyclohexylphenyl)-2-(4-fluorophenyl)-N-methylimidazo[1,2-a] |
| | pyrimidine-3-carboxamide |
| 143 | 2-(4-fluorophenyl)-N-methyl-N-[4-(morpholin-4-yl)phenyl]imidazo[1,2-a] |
| | pyrimidine-3-carboxamide |
| 144 | N-(biphenyl-3-yl)-2-(4-fluorophenyl)-N-methylimidazo[1,2-a]pyrimidine-3- |
| | carboxamide |
| 145 | N-(4-chlorophenyl)-2-(4-fluoro-3-methoxyphenyl)-N-methylimidazo[1,2-a] |
| | pyrimidine-3-carboxamide |
| 146 | N-(4-chlorophenyl)-N-methyl-2-{4-[(phenylsulfonyl)amino]phenyl} |
| | imidazo[1,2-a]pyrimidine-3-carboxamide |
| 147 | methyl (4-{3-[(4-chlorophenyl)(methyl)carbamoyl]imidazo[1,2-a]pyrimidin-2- |
| | yl}phenyl)carbamate |
| 148 | N-(4-chlorophenyl)-N-methyl-2-{4-[(methylcarbamoyl)amino]phenyl}imidazo |
| | [1,2-a]pyrimidine-3-carboxamide |
| 149 | N-(4-chlorophenyl)-N-methyl-2-{4-[(phenylsulfamoyl)amino]phenyl}imidazo |
| | [1,2-a]pyrimidine-3-carboxamide |
| 150 | N-(4-chlorophenyl)-2-[4-(4-fluorophenoxy)phenyl]-N-methylimidazo[1,2-a] |
| | pyrimidine-3-carboxamide |
| 151 | N-(4-chlorophenyl)-N-methyl-2-[4-(methylsulfanyl)phenyl]imidazo[1,2-a] |
| | pyrimidine-3-carboxamide |
| 152 | N-(4-chlorophenyl)-2-{4-[(4-fluorophenyl)amino]phenyl}-N-methylimidazo |
| | [1,2-a]pyrimidine-3-carboxamide |
| 153 | N-(4-chlorophenyl)-N-methyl-2-[4-(piperazin-1-ylcarbonyl)phenyl]imidazo |
| | [1,2-a]pyrimidine-3-carboxamide |
| 154 | N-(4-chlorophenyl)-N-methyl-2-{4- |
| | [methyl(phenyl)carbamoyl]phenyl}imidazo[1,2-a]pyrimidine-3-carboxamide |
| 155 | N-(4-chlorophenyl)-2-{4-[(4-fluorophenyl)sulfonyl]phenyl}-N-methylimidazo |
| | [1,2-a]pyrimidine-3-carboxamide |
| 156 | N-(4-chlorophenyl)-2-{4-[(4-fluorophenyl)sulfamoyl]phenyl}-N-methyl |
| | imidazo[1,2-a]pyrimidine-3-carboxamide |
| 157 | N-[4-(4-chlorophenoxy)phenyl]-2-(4-fluorophenyl)-N-methylimidazo[1,2-a] |
| | pyrimidine-3-carboxamide |
| 158 | N-{4-[(4-chlorophenyl)sulfanyl]phenyl}-2-(4-fluorophenyl)-N-methylimidazo |
| | [1,2-a]pyrimidine-3-carboxamide |
| 159 | N-methyl-2-phenyl-N-[4-(phenylsulfamoyl)phenyl]imidazo[1,2-a]pyrimidine- |
| | 3-carboxamide |
| 160 | N-(4-chlorophenyl)-2-(4-fluorophenyl)-N-methyl-6-[(phenylcarbonyl)amino] |
| | imidazo[1,2-a]pyrimidine-3-carboxamide |
| 161 | N-(4-chlorophenyl)-2-(4-fluorophenyl)-N-methyl-6-[(phenylsulfonyl)amino] |
| | imidazo[1,2-a]pyrimidine-3-carboxamide |
| 162 | N-(4-chlorophenyl)-2-(4-fluorophenyl)-N-methyl-6-[(phenylcarbamoyl)amino] |
| | imidazo[1,2-a]pyrimidine-3-carboxamide |
| 163 | N-(4-chlorophenyl)-2-(4-fluorophenyl)-6-hydroxy-N-methylimidazo[1,2-a] |
| | pyrimidine-3-carboxamide |
| 164 | N-(4-chlorophenyl)-2-(4-fluorophenyl)-N-methyl-6-(phenylsulfamoyl)imidazo |
| | [1,2-a]pyrimidine-3-carboxamide |
| 165 | N-(4-chlorophenyl)-2-(4-fluorophenoxy)-N-methylimidazo[1,2-a]pyrimidine- |
| | 3-carboxamide 4-methylbenzenesulfonate (1:1) |
| 166 | N-(4-chlorophenyl)-2-(4-fluorophenoxy)-N-methylimidazo[1,2-a]pyrimidine- |
| | 3-carboxamide benzenesulfonate (1:1) |
| 167 | N-(4-chlorophenyl)-2-(4-fluorophenoxy)-6-hydroxy-N-methylimidazo[1,2-a] |
| | pyrimidine-3-carboxamide |
| 168 | N-(4-chlorophenyl)-2-(4-fluorophenoxy)-5-hydroxy-N-methylimidazo[1,2-a] |
| | pyrimidine-3-carboxamide |
| 169 | N-(4-chlorophenyl)-2-(4-fluorophenoxy)-7-hydroxy-N-methylimidazo[1,2-a] |
| | pyrimidine-3-carboxamide |
| 170 | N-(4-chlorophenyl)-2-(4-fluorophenoxy)-6-hydroxyimidazo[1,2-a]pyrimidine- |
| | 3-carboxamide |
| 171 | N-(4-chlorophenyl)-2-(4-fluorophenoxy)-5-hydroxyimidazo[1,2-a]pyrimidine- |
| | 3-carboxamide |
| 172 | N-(4-chlorophenyl)-2-(4-fluorophenoxy)-7-hydroxyimidazo[1,2-a]pyrimidine- |
| | 3-carboxamide |
| 173 | N-(4-chlorophenyl)-2-(4-fluorophenoxy)-N-(hydroxymethyl)imidazo[1,2-a] |
| | pyrimidine-3-carboxamide |
| 174 | N-(4-chlorophenyl)-2-(4-fluoro-3-hydroxyphenoxy)-N-methylimidazo[1,2-a] |
| | pyrimidine-3-carboxamide |
| 175 | N-(4-chlorophenyl)-2-(4-fluoro-3-hydroxyphenoxy)imidazo[1,2-a]pyrimidine- |
| | 3-carboxamide |
| 176 | N-(4-chloro-3-hydroxyphenyl)-2-(4-fluorophenoxy)-N-methylimidazo[1,2-a] |
| | pyrimidine-3-carboxamide |
| 177 | N-(4-chloro-3-hydroxyphenyl)-2-(4-fluorophenoxy)imidazo[1,2-a]pyrimidine- |
| | 3-carboxamide |
| 178 | 2-(4-fluorophenoxy)imidazo[1,2-a]pyrimidine-3-carboxamide |

In preferred embodiment, the present invention provides a compound selected from the group comprising of:

| Comp | |
|---|---|
| No. | Chemical Name |
| 1 | N-(4-chlorophenyl)-N-methyl-2-phenylimidazo[1,2-a]pyrimidine-3- |
| | carboxamide; |
| 7 | N-(4-chlorophenyl)-2-(4-fluorophenyl)-N-methylimidazo[1,2-a]pyrimidine-3- |
| | carboxamide; |
| 20 | N-(4-chlorophenyl)-N-methyl-2-(pyridin-3-yl) imidazo[1,2-a]pyrimidine-3- |
| | carboxamide; |
| 22 | N, 2-bis(4-fluorophenyl)-N-methylimidazo[1,2-a]pyrimidine-3-carboxamide; |
| 30 | N-(4-chlorophenyl)-2-(4-hydroxyphenyl)-N-methylimidazo[1,2-a]pyrimidine- |
| | 3-carboxamide; |
| 33 | N-(4-fluorophenyl)-N-methyl-2-[3-(trifluoromethyl) phenyl]imidazo[1,2- |
| | a]pyrimidine-3-carboxamide; |
| 50 | N-(4-chlorophenyl)-2-(4-fluorophenoxy)-N-methylimidazo[1,2-a]pyrimidine- |
| | 3-carboxamide; |
| 45 | N-(3-chloro-4-fluorophenyl)-2-(3-fluoro-4-methoxyphenyl)-N- |
| | methylimidazo[1,2-a]pyrimidine-3-carboxamide; |
| 136 | N-(4-chlorophenyl)-N-cyclopropyl-2-(4-fluorophenyl)imidazo[1,2-a] |
| | pyrimidine-3-carboxamide; and |
| 65 | N-(4-chlorophenyl)-2-(4-fluorophenyl)-N-(propan-2-yl)imidazo[1,2-a] |
| | pyrimidine-3-carboxamide or pharmaceutically acceptable salts thereof. |

In another embodiment, present invention provides the use of compound of formula (I) for the preparation of salts, isomers, stereoisomers, conformers, tautomers, polymorphs, hydrates and solvates of compound of formula (I).

In preferred embodiment present invention provides the use of compound of formula (I) for the preparation of salts, polymorphs, hydrates and solvates of compound of formula (I).

In another embodiment, present invention provides the process of preparation of polymorph of compound of formula (I), comprises contacting compound of formula (I) with suitable solvent or mixture of solvent.

Another embodiment of the present invention provides the process of preparation of salt of compound of formula (I), comprises contacting compound of formula (I) with suitable acid or base, optionally in the presence of suitable solvent or mixture of solvent.

### DEFINITIONS

The following definitions apply to the terms as used throughout this specification, unless otherwise limited in specific instances:

The term “compound” employed herein refers to any compound encompassed by the generic formula disclosed herein. The compounds described herein may contain one or more double bonds and therefore, may exist as isomers, stereoisomers, such as geometric isomers, E and Z isomers, and may possess asymmetric carbon atoms (optical centers) and therefore may exist as enantiomers, diastereoisomers. Accordingly, the chemical structures described herein encompasses all possible stereoisomers of the illustrated compounds including the stereoisomerically pure form (e.g., geometrically pure) and stereoisomeric mixtures (racemates). The compound described herein, may exist as a conformational isomers such as chair or boat form. The compounds may also exist in several tautomeric forms including the enol form, the keto form and mixtures thereof. Accordingly, the chemical structures described herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds of the invention include, but are not limited to ²H, ³H, ¹³C, ¹⁴C, ¹⁵N, ¹⁸O, ¹⁷O, etc. Compounds may exist in non-solvated forms as well as solvated forms, including hydrated forms. In general, compounds may be hydrated or solvated. Certain compounds may exist in multiple crystalline or amorphous forms. The polymorphic forms can be prepared by the techniques known in the art. Preferably, a compound of formula (I) is treated with suitable solvent or mixture of solvents at suitable temperature to produce a polymorphic form of compound of formula (I). The polymorphic form of compound of formula (I) is crystalline or amorphous form and can be characterized by techniques known in the art such as XRPD or IR spectrum. In general, all physical forms are equivalent for the uses contemplated herein and are intended to be within the scope and spirit of the present invention.

The use of the terms “a” and “an” and “the” and similar referents in the context of describing the invention (especially in the context of the following claims) are to be construed to cover both the singular and the plural, unless otherwise indicated herein or clearly contradicted by context.

Further, it should be understood, when partial structures of the compounds are illustrated, a dash (“-”) indicate the point of attachment of the partial structure to the rest of the molecule.

The nomenclature of the compounds of the present invention as indicated herein is according to ACD LABS/Chemsketch® (Product version: 12) IUPAC NAME.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, carbonic acid, phosphoric acid, and the like; or formed with organic acids such as acetic acid, propionic acid, isobutyric acid, hexanoic acid, cyclopentanepropionic acid, oxalic acid, glycolic acid, pyruvic acid, lactic acid, malonic acid, succinic acid, suberic acid, malic acid, maleic acid, fumaric acid, tartaric acid, citric acid, benzoic acid, 3-(4-hydroxybenzoyl) benzoic acid, phthalic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid or 4-methylbenzenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glucuronic acid, galactunoric acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like. Also included are salts of amino acids such as arginate and the like (see, for example, Berge, S. M., et al., “Pharmaceutical Salts”, Journal of Pharmaceutical Science, 1977, 66, 1-19).

As used herein, the term “polymorphs” pertains to compounds having the same chemical formula, the same salt type and having the same form of hydrate/solvate but having different crystallographic properties.

As used herein, the term “hydrates” pertains to a compound having a number of water molecules bonded to the compound.

As used herein, the term “solvates” pertains to a compound having a number of solvent molecules bonded to the compound.

The term “substituted”, as used herein, includes mono- and poly-substitution by a named substituent to the extent such single and multiple substitution (including multiple substitution at the same site) is chemically allowed and which means that any one or more hydrogen on the designated atom is replaced with a selection from the indicated group, provided that the designated atom's normal valence is not exceeded, and that the substitution results in a stable compound, for example, when a substituent is keto, then the two hydrogen on the atom are replaced. All substituents (R, R₁, R₂ . . . ) and their further substituents described herein may be attached to the main structure at any heteroatom or carbon atom which results in formation of stable compound.

As used herein, a “halo” or “halogen” substituent is a monovalent halogen radical chosen from chloro, bromo, iodo and fluoro.

The term “alkyl” or “Alk” used either alone or in attachment with another group refers to a cyclic, branched, or straight chain saturated aliphatic hydrocarbon radical, which may be optionally substituted. When a subscript is used with reference to an alkyl, the subscript refers to the number of carbon atoms that group may contain. For example, a “C₁-C₈” would refer to any alkyl group containing one to eight carbons in the structure. Alkyl may be straight chain, branched chain or cyclic. The said alkyl or “alk” may be optionally substituted with one or more substituent independently selected from the group consisting of —OH, —SH, —COOH, -oxo, -thioxo, -halo, -amino, -mono(C₁₋₃alkyl)amino, -di(C₁₋₃alkyl)amino, —S(C₁₋₃alkyl), -aryl, -heteroaryl and —C₁₋₃ alkoxy.

The term “alkoxy” refers to any alkyl group as defined herein above attached to the parent molecular moiety through an oxygen bridge.

The term “aryl” refers to an aromatic group which is a 6 to 10 membered monocyclic or bicyclic carbon-containing ring system, which may be unsubstituted or substituted. The said aryl may be optionally substituted with one or more substituent independently selected from the group consisting of halo, cyano, —N(R₄)(R₄), —OH, —OC₁₋₈ alkyl, —OCF3, —CF3, —NO2, —SC₁₋₈alkyl, —S(O₂)C₁₋₈alkyl, —COOH, —CON(R₄)(R₄), wherein R₄ is as defined herein above.

The term “amine” refers to NH2, which is optionally substituted by one or more alkyl, aryl, heteroaryl, heterocyclyl, urea or carbamate.

The term “cycloalkenyl” refers to a monovalent group derived from a monocyclic or bicyclic unsaturated carbocyclic ring compound containing between three and twenty carbon atoms by removal of a single hydrogen atom.

The term “heteroaryl” refers to an aromatic group, which is a 5 to 10 membered monocyclic or bicyclic ring system, which has at least one heteroatom, which may be unsubstituted or substituted. The term “heteroatom” as used herein includes oxygen, sulfur and nitrogen. The said heteroaryl may be optionally substituted with one or more substituent independently selected from the group consisting of halo, cyano, —N(R₄)(R₄), —OH, —OC₁₋₈ alkyl, —OCF3, —CF3, —NO2, —SC₁₋₈alkyl, —S(O₂)C₁₋₈alkyl, —COOH, —CON(R₄)(R₄), wherein R₄ is as defined herein above.

The term “heterocyclyl” refers to a fully or partially saturated cyclic group, which is a 5 to 10 membered monocyclic or bicyclic ring system, which has at least one heteroatom, which may be unsubstituted or substituted. The term “heteroatom” as used herein includes oxygen, sulfur and nitrogen. The said heterocyclyl may be optionally substituted with one or more substituent independently selected from the group consisting of halo, cyano, —N(R₄)(R₄), —OH, —OC₁₋₈ alkyl, —OCF3, —CF3, —NO2, —SC₁₋₈alkyl, —S(O₂)C₁₋₈alkyl, —COOH, —CON(R₄)(R₄), wherein R₄ is as defined herein above.

As used herein the term “contacting” includes coming together, to bring or put in contact, mixing, interacting, reacting, suspending, dissolving or more than one act as mentioned.

As used herein, the term “mammal” means a human or an animal such as monkeys, primates, dogs, cats, horses, cows, etc.

The terms “treating” or “treatment” of any disease or disorder as used herein to mean administering a compound to a mammal in need thereof. The compound may be administered to provide a prophylactic effect in terms of completely or partially preventing or delaying the onset of a disease or disorder or sign or symptom thereof; and/or the compound may be administered to provide a partial or complete cure for a disease or disorder and/or sign or symptom attributable to the disease or disorder.

The phrase “a therapeutically effective amount” means the amount of a compound that, when administered to a patient for treating a disease, is sufficient to effect such treatment for the disease. The “therapeutically effective amount” will vary depending on the compound, mode of administration, the disease and its severity and the age, weight, etc., of the patient to be treated.

Throughout this specification and the appended claims it is to be understood that the words “comprise” and “include” and variations such as “comprises”, “comprising”, “includes”, “including” are to be interpreted inclusively, unless the context requires otherwise. That is, the use of these words may imply the inclusion of an element or elements not specifically recited.

In another embodiment, present invention provides the process for preparing the compounds of formula (I).

The following reaction schemes are given to disclose the synthesis of the compounds according to the present invention.

The compound of formula (I) can be prepared by the following methods described in schemes I & II.

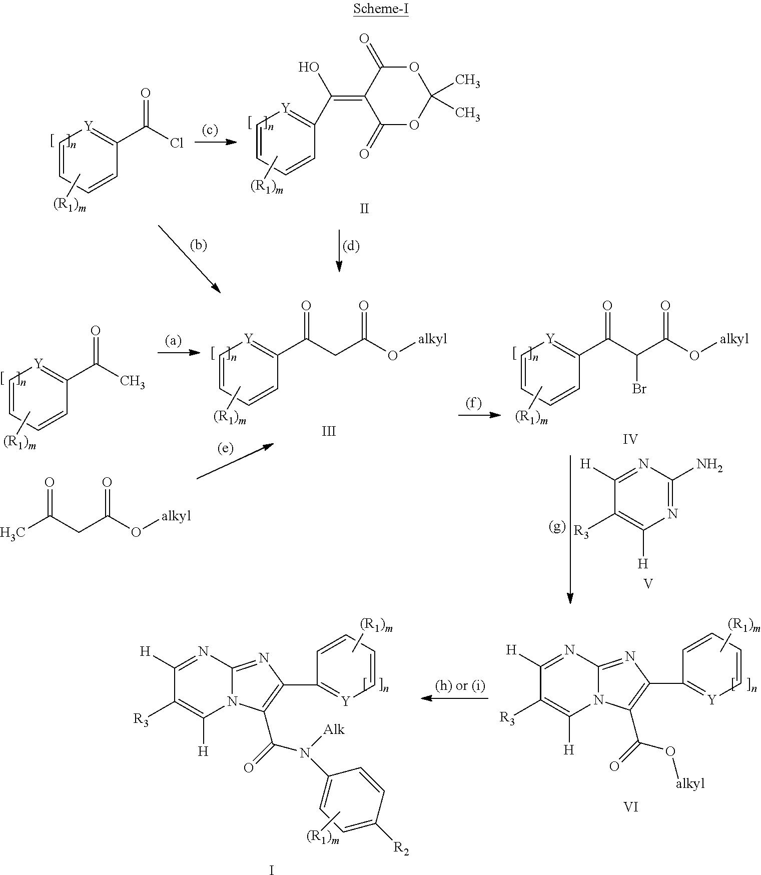

In Step (h), the compound of formula (I) can be prepared by reacting the compound of the formula (VI) with the appropriate secondary amine in the presence of inorganic or organic base such as triethylamine, potassium carbonate, sodium ethoxide, potassium tert-butoxide or 1′8-diazabicyclo[5,4,0]undec-7-ene in the polar protic or non-polar aprotic solvent like toluene, xylene, ethanol, acetonitrile or N,N-Dimethyl formamide at a temperature in the range of 80° C. to 130° C. for 4 h to 10 h to give the compound of formula (I).

In alternate way, the compound of formula (I) can be prepared by reacting the compound of the formula (VI) with the appropriate primary amine in the presence of inorganic or organic base such as triethylamine, potassium carbonate, sodium ethoxide, potassium tert-butoxide or 1′ 8-diazabicyclo[5,4,0]undec-7-ene in the polar protic or non-polar aprotic solvent like toluene, xylene, ethanol, acetonitrile or N,N-Dimethyl formamide at a temperature in the range of 80° C. to 130° C. for 4 h to 10 h to give the corresponding amide derivative. The obtained amide derivatives is reacted with appropriate alkyl halide in the presence of inorganic or organic base such as N-ethyldiisopropylamine, triethylamine, cesium carbonate or potassium carbonate in polar protic or non-polar aprotic solvent like tetrahydrofuran, N, N-dimethyl formamide or acetonitrile at a temperature in the range of 0° C. to 60° C. for a period of 30 min to 4 h to give the compound of formula (I).

In Step (i), alternatively, the compound of the formula (VI) is treated with inorganic base such as sodium hydroxide or potassium hydroxide in a polar protic solvent like ethanol, methanol, isopropanol at the temperature in the range of 0° C. to 60° C. for 1 h to 12 h to give the corresponding carboxylic acid, which is further treated with N-ethyldiisopropylamine, 1-hydroxybenzotraizole and 1-(3-dimethylaminopropyl)-3-ethylcarbodiimide hydrochloride (EDCI) or benzotriazole-1-yl-oxytris(dimethylamino)phosphonium hexafluorophosphate (BOP); or with thionyl chloride or oxalyl chloride in the presence of catalytic amount of dimethylformamide in a polar protic or non-polar aprotic solvent such as tetrahydrofuran, acetonitrile or N,N Dimethyl formamide at temperature from 0° C. to 60° C. for 1 h to 3 h, then the appropriate amine is added and stirred at room temperature in the presence of inorganic or organic base like triethylamine or potassium carbonate for 1 h to 12 h to give the compound of formula (I).

In an alternate way, the corresponding carboxylic acid as obtained in step (i) is treated with alkyl chloroformate in the presence of organic or inorganic base such as N-ethyldiisopropylamine, triethylamine or potassium carbonate in a solvent like tetrahydrofuran, acetonitrile or toluene at room temperature for a period of 1 h to 4 h to give the mixed anhydride, which is further reacted with the appropriate amine at the temperature in the range of 0° C. to 110° C. for a period of 1 h to 6 h to give the compound of formula (I).

In step (g), the compound of the formula (VI) is prepared by reacting the compound of formula (IV) with formula (V) in the polar protic or aprotic solvent like methanol, ethanol, isopropanol, N, N-dimethylformamide or N-methyl 2-pyrrolidinone at the temperature in the range of 90° C. to 140° C. for 4-12 h.

In step (f), the compound of the formula (IV) is prepared by reacting the compound of formula (III) with suitable halogenating reagents such as N-bromosuccinimide or N-chlorosuccinimide in the presence of catalyst such as ammonium acetate and in the suitable solvent like diethyl ether, diisopropyl ether or 1,4-dioxane at the room temperature 1-12 hour.

In step (e), the compound of formula (III) is prepared by reacting the acid chloride with alkyl acetoacetate in the presence of inorganic or organic base such as pyridine, sodium ethoxide, sodium hydroxide or anhydrous magnesium chloride and in the non-polar aprotic or polar aprotic solvent like toluene, tetrahydrofuran under inert atmosphere at the temperature in the range of 0° C. to 60° C. for 1-12 hour. Further, the product is treated with suitable base like sodium hydroxide or potassium hydroxide in the alcoholic solvent like ethanol, methanol or isopropanol.

In step (c, d), alternatively, the compound of formula (III) is prepared by reacting the acid chloride with isopropylidene malonate (Meldrum's acid) in the presence of inorganic or organic base such as triethylamine, pyridine, sodium ethoxide, sodium hydroxide or anhydrous magnesium chloride and in the non-polar aprotic or polar aprotic solvent like dichloromethane, toluene, tetrahydrofuran under inert atmosphere at the temperature in the range of 0° C. to 60° C. for 1-12 h to give the compound of formula (II), which is refluxed in alcoholic solvent such as methanol or ethanol.

In step (b), alternatively, the compound of formula (III) is prepared by reacting acid chloride with alkyl acetate in the presence of suitable base such as sodium hydride or lithium bis(trimethylsilyl)amide in the non-polar aprotic or polar aprotic solvent like toluene, tetrahydrofuran or dimethylformamide under inert atmosphere at the temperature in the range of −20° C. to 60° C. for 1-6 hour.

In step (a), alternatively, the compound of formula (III) is prepared by reacting acetophenone derivative with the dialkyl carbonate or alkyl chloroformate in the presence of suitable base such as sodium hydride, potassium tert-butoxide or lithium bis (trimethylsilyl) amide in the non-polar aprotic or polar aprotic solvent like toluene, tetrahydrofuran, dimethylformamide and N-methyl pyrrolidinone under inert atmosphere at the temperature in the range of −20° C. to 100° C. for 1-12 hour.

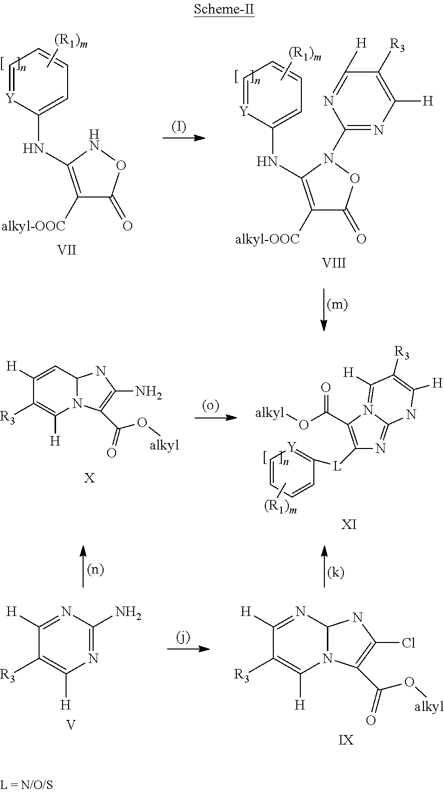

In step (k), the compound of formula (XI) is prepared by reacting the compound of formula (IX) with the appropriate phenol, thiophenol and aniline in the presence of inorganic or organic base such as potassium carbonate, sodium carbonate, triethylamine or cesium carbonate in the non-polar aprotic or polar aprotic solvent like toluene, tetrahydrofuran, dimethylformamide, N, N-dimethylacetamide and N-methyl pyrrolidinone at the temperature in the range of 30° C. to 140° C. for 1-12 hour.

The compound of formula (I) can be prepared from compound of formula (XI) in analogues manner as described in scheme I.

In step (j), the compound of the formula (IX) is prepared by reacting the compound of formula (V) with dialkyl bromomalonate in the polar protic or polar aprotic solvent like methanol, ethanol, isopropanol, N, N-dimethylformamide or N-methyl 2-pyrrolidinone at the temperature in the range of 90° C. to 140° C. for 4-12 hr to give the hydroxy-cyclized product, which is treated with phosphorus oxychloride in the aprotic solvent like toluene, tetrahydrofuran, under inert atmosphere at the temperature in the range of 20° C. to 100° C. for 1-12 hour.

In step (m), the compound of formula (XI) is prepared by reacting the compound of the formula (VIII) with triethylamine in the polar protic solvent like ethanol, isopropanol at the temperature in the range of 30° C. to 140° C. for 1-12 hour.

In step (o), the compound of the formula (XI) is prepared by reacting the compound of formula (X) with the aryl boronic acid in the non-polar aprotic solvent like 1,2-dichloroethane or dichloromethane in the presence of base like triethylamine or pyridine using copper acetate as a catalyst at the temperature in the range of 30° C. to 80° C. for 4-12 hr.

In step (n), the compound of the formula (X) is prepared by reacting the compound of formula (V) with alkyl cyanoacetate such as ethyl cyanoacetate in the polar protic or polar aprotic solvent like methanol, ethanol, isopropanol, N, N-dimethylformamide or N-methyl 2-pyrrolidinone at the temperature in the range of 90° C. to 140° C. for 4-12 hr to give the amino-cyclized product.

In step (l), the compound of formula (VIII) is prepared by treating the compound of the formula (VII) with 2-chloropyrimidine derivatives under inert atmosphere at the temperature in the range of 100° C. to 140° C. for 1-4 h. The compound of the formula (VII) is prepared by procedure given in the literature (ARKIVOC 2005 (xiv), 59-70).

A general synthetic method is provided for each of the disclosed groups of chemical compounds. One of ordinary skill will recognize to substitute appropriately modified starting material containing the various substituents. One of ordinary skill will readily synthesize the disclosed compounds according to the present invention using conventional synthetic organic techniques and microwave techniques from starting material which are either purchased or may be readily prepared using known methods.

The novel compounds of the present invention were prepared according to the procedure of the schemes as described herein above, using appropriate materials and are further exemplified by the following specific examples. The examples are not to be considered nor construed as limiting the scope of the invention set forth.

### EXAMPLES

### Example 1

### Preparation of N-(4-chlorophenyl)-2-(4-fluorophenyl) imidazo[1,2-a]pyrimidine-3-carboxamide (Compound No. 32)

### Step A: Preparation of ethyl 3-(4-fluorophenyl)-3-oxopropanoate

To a stirred solution of 4-fluoroacetophenone (20 g, 144 mmol) and diethyl carbonate (85 ml, 720 mmol), sodium hydride (6.9 g, 144 mmol) was added portion wise at temperature (0° C.-5° C.) under nitrogen atmosphere in 1 hour. The reaction mixture was heated to 60° C. and stirred for 30 minutes. The reaction mixture was cooled to 0° C. and was poured into ice cold water (150 ml) and extracted with dichloromethane (2×100 ml). The combined organic layer was dried over sodium sulphate, concentrated under vacuo to give 30 g of the titled product as brown viscous oil.

¹H NMR (400 MHz, DMSO-d₆) δ: 1.15-1.18 (3H, t), 4.10-4.14 (2H, q), 4.20 (2H, s), 7.61-7.64 (2H, d), 7.94-7.97 (2H, d).

m/z=211 (M+H)⁺

### Step B: Preparation of ethyl 2-bromo-3-(4-fluorophenyl)-3-oxopropanoate

To a stirred solution of ethyl 3-(4-fluorophenyl)-3-oxopropanoate (30 g, 144 mmol) in diethyl ether (150 ml), N-Bromo succinimide (25.2 g, 144 mmol) was added portionwise at 5-10° C. followed by addition of ammonium acetate (2.2 g, 28.8 mmol). The reaction mixture was stirred at room temperature (25° C.-27° C.) for 4 h. The reaction mixture was filtered and the filtrate was washed with aqueous sodium bicarbonate solution (2×50 ml) and finally with water. The organic layer was dried over sodium sulfate and concentrated under vacuo to give 30 g of the titled product as brown viscous oil.

¹H NMR (400 MHz, DMSO-d₆) δ: 1.16 (3H, t), 4.20-4.23 (2H, q), 6.68 (1H, s), 7.43-7.45 (2H, m), 8.10-8.14 (2H, m).

m/z=289, 291 (M+2H)⁺

### Step C: Preparation of ethyl 2-(4-fluorophenyl) imidazo[1,2-a]pyrimidine-3-carboxylate

To a stirred solution of ethyl 2-bromo-3-(4-fluorophenyl)-3-oxopropanoate (30 g, 104 mmol) in isopropyl alcohol (150 ml), 2-aminopyrimidine (9.8 g, 104 mmol) was added and further stirred for 6 hours at 90° C. The reaction mixture was cooled to 30° C. and isopropyl alcohol was removed. The crude product was stirred in cold ethyl acetate (50 ml) and solid was filtered to give the titled compound as brown solid (35 g).

¹H NMR (400 MHz, DMSO-d₆) δ: 1.20-1.24 (3H, t), 4.27-4.32 (2H, q), 7.30-7.34 (2H, t), 7.37-7.39 (1H, q), 7.87-7.91 (2H, m), 8.80-8.82 (1H, dd), 9.59-9.61 (1H, dd).

m/z=286 (M+H)⁺

### Step D: Preparation of 2-(4-fluorophenyl) imidazo[1,2-a]pyrimidine-3-carboxylic acid

To a stirred solution of Ethyl 2-(4-fluorophenyl)imidazo[1,2-a]pyrimidine-3-carboxylate 35 g (120 mmol) in methanol (150 ml), aqueous solution of sodium hydroxide (9.8 g, 240 mmol in 50 ml water) was added slowly at 10° C. and stirred 4 hours at room temperature (30-32° C.). The reaction mixture was quenched with water (100 ml). Methanol was removed under vacuo at 40° C. and aqueous layer was washed with ethyl acetate (2×200 ml). The aqueous layer was acidified to pH 3-4 with 2N hydrochloric acid and the separated solid was filtered, washed with water, dried under vacuo to give 19 g of the titled compound as brown solid.

¹H NMR (400 MHz, DMSO-d₆) δ: 7.29-7.39 (4H, m), 7.89-7.92 (2H, t), 8.77-8.79 (1H, dd), 9.65-9.67 (1H, dd), 13.4 (1H, bs).

m/z=258 (M+H)⁺

### Step E: Preparation of N-(4-chlorophenyl)-2-(4-fluorophenyl) imidazo[1,2-a]pyrimidine-3-carboxamide

To a stirred solution of 2-(4-fluorophenyl) imidazo[1,2-a]pyrimidine-3-carboxylic acid (11 g, 40 mmol) in toluene (100 ml), thionyl chloride (6.3 ml, 80 mmol) was added under nitrogen atmosphere, followed by 2-3 drops of N, N Dimethylformamide. The reaction mixture was stirred for 2 hour at 60° C. The reaction mixture was concentrated under vacuo and the obtained crude product was dissolved in dichloromethane (80 ml) and added drop wise to the solution of 4-chloroaniline (5.1 g, 40 mmol) and triethylamine (17 ml, 120 mmol) in dichloromethane (20 ml) at 0° C. The reaction mixture was stirred at room temperature (28-30° C.) for 4 hours and the separated solid was filtered, washed with water (2×30 ml), saturated sodium bicarbonate solution (2×20 ml) and ethyl acetate (2×30 ml). Then the solid was dried under vacuo to give 9.0 g of desired compound as an orange color solid.

¹H NMR (400 MHz, DMSO-d₆) δ: 7.22-7.25 (1H, q), 7.29-7.33 (2H, t), 7.39-7.41 (2H, d), 7.62-7.64 (2H, d), 7.94-7.97 (2H, q), 8.70-8.72 (1H, dd), 9.25-9.27 (1H, dd), 10.50 (1H, bs).

m/z=367 (M+H)⁺

### Example 2

### Preparation of N-(4-chlorophenyl)-2-(4-fluorophenyl)-N-methylimidazo[1,2-a]pyrimidine-3-carboxamide (Compound no. 7)

To a stirred solution of N-(4-chlorophenyl)-2-(4-fluorophenyl) imidazo[1,2-a]pyrimidine-3-carboxamide (9 g, 24 mmol) (as prepared in Example-1) in N,N dimethylformamide (45 ml), cesium carbonate (15.9 g, 48 mmol) was added portion wise and stirred for 30 min, followed by addition of iodomethane (10.34 g, 72 mmol) at 0-10° C. The reaction mixture was stirred for 4 h at 10° C. Reaction mixture was poured in ice water under stirring. The obtained solid was filtered, washed with water (2×30 ml) and dried under vacuum to give 7.0 g of desired compound as light orange color solid.

(CDCl3) δ: 3.41 (3H, s), 6.32-6.34 (2H, m), 6.85-6.88 (2H, d), 7.03-7.07 (3H, m), 7.33-7.37 (2H, m), 8.66-8.67 (1H, m), 9.01-9.03 (1H, dd).

m/z=381 (M+H)⁺

### Example 3

### Preparation of N-(4-chlorophenyl)-N-methyl-2-(pyridin-2-yl)imidazo[1,2-a]pyrimi dine-3-carboxamide (Compound no. 11)

### Step A: Preparation of ethyl 3-oxo-3-(pyridin-2-yl) propanoate

The titled compound was prepared in analogous manner as described in Step-A of example 1.

¹H NMR (400 MHz, DMSO-d₆) δ: 1.22-1.24 (3H, t), 4.17-4.22 (2H, q), 4.21 (2H, s), 7.48-7.51 (1H, m), 7.81-7.86 (1H, m), 8.07-8.09 (1H, dd), 8.64-8.66 (1H, dd).

m/z=194 (M+H)⁺

### Step B: Preparation of ethyl 2-bromo-3-oxo-3-(pyridin-2-yl) propanoate

The titled compound was prepared in analogous manner as described in Step-B of example 1.

¹H NMR (400 MHz, DMSO-d₆) δ: 1.12-1.16 (3H, t), 4.18-4.23 (2H, q), 6.42 (1H, s), 7.70-7.73 (1H, q), 8.07-8.08 (2H, d), 8.71-8.72 (1H, dd).

m/z=272 (M+H)⁺, 274 (M+2H)⁺

### Step C: Preparation of ethyl 2-(pyridin-2-yl) imidazo[1,2-a]pyrimidine-3-carboxylate

The titled compound was prepared in analogous manner as described in Step-C of example 1.

¹H NMR (400 MHz, DMSO-d₆) δ: 1.08-1.12 (3H, t), 4.19-4.25 (2H, q), 7.39-7.40 (1H, m), 7.80-7.82 (1H, dd), 8.68-8.70 (1H, d), 8.82-8.84 (1H, dd), 9.50-9.52 (1H, dd).

m/z=269 (M+H)⁺

### Step D: Preparation of 2-(pyridin-2-yl) imidazo[1,2-a]pyrimidine-3-carboxylic acid

The titled compound was prepared in analogous manner as described in Step-D of example 1.

¹H NMR (400 MHz, DMSO-d₆) δ: 7.29 (1H, m), 7.61 (1H, dd), 8.13 (1H, m), 8.42-8.55 (1H, m), 8.76 (2H, s), 10.00-10.02 (1H, d).

m/z=239 (M−H)+

### Step E: Preparation of N-(4-chlorophenyl)-2-(pyridin-2-yl) imidazo[1,2-a]pyrimidine-3-carboxamide

To a stirred solution of 2-(pyridin-2-yl) imidazo[1,2-a]pyrimidine-3-carboxylic acid (0.5 g, 2.1 mmol) in dichloromethane (20 ml), N-disopropylethylamine (1.1 ml, 6 mmol), 1-hydroxy benzotriazole (0.56 g, 4.2 mmol) and 1-ethyl-3-(3-dimethylamino) propylcarbdiimide hydrochloride (0.768 g, 4.2 mmol) was added at 10° C. and stirred for 30 min, followed by addition of 4-chloroaniline (0.33 g, 2.2 mmol). The reaction mixture was stirred at room temperature for 4 hour. The reaction mixture was concentrated under vacuo, water (20 ml) was added and the precipitated solid was filtered and dried under vacuo to give 0.35 g of the desired compound as yellow color solid.

¹H NMR (400 MHz, CDCl₃) δ: 7.10 (1H, s), 7.27 (2H, s), 7.37-7.39 (1H, d), 7.51 (1H, s), 7.77-7.79 (2H, d), 8.03 (1H, d), 8.75-8.82 (2H, d), 10.34 (1H, s), 14.95 (1H, s).

m/z=350 (M+H)⁺

### Step F: Preparation of N-(4-chlorophenyl)-N-methyl-2-(pyridin-2-yl) imidazo[1,2-a]pyrimidine-3-carboxamide

To a stirred solution of N-(4-chlorophenyl)-N-methyl-2-(pyridin-2-yl) imidazo[1,2-a]pyrimidine-3-carboxamide (0.35 g, 1 mmol) in tetrahydrofuran (10 ml), sodium hydride (0.1 g, 2 mmol) was added portion wise and stirred for 30 min, followed by addition of iodomethane (0.28 g, 2 mmol) at 10-15° C. The reaction mixture was stirred for 4 h at 10° C. The reaction mixture was concentrated under vacuo and ice water was added, extracted with dichloromethane (3×100 ml) and dried over sodium sulphate. The crude product was purified on column chromatography using 30% ethyl acetate in hexane as eluent to give 0.2 g of the titled compound as light yellow solid.

¹H NMR (400 MHz, DMSO-d₆) δ: 3.39 (3H, s), 6.98-7.04 (4H, m), 7.20-7.23 (1H, m), 7.38-7.41 (1H, t), 7.76 (1H, m), 7.82-7.84 (1H, t), 8.65-8.67 (1H, m), 8.68-8.69 (1H, d), 8.98-9.0 (1H, d).

m/z=364 (M+H)⁺

### Example 4

### Preparation of N-(2,4-difluorophenyl)-2-(4-fluorophenoxy)-N-methylimidazo[1,2-a]pyrimidine-3-carboxamide (Compound no. 46)

### Step A: Preparation of ethyl 2-hydroxyimidazo[1,2-a]pyrimidine-3-carboxylate

To a stirred solution of 2-aminopyrimidine(40 g, 421 mmol) in ethanol (200 ml), diethyl bromomalonate (125.7 g, 526 mmol) was added and refluxed for 24 h. The reaction mixture was concentrated under vacuo and ethyl acetate (100 ml) was added. The reaction mixture was stirred for 30 min and separated solid was filtered, washed with hexane (2×50 ml) and dried under vacuo to yield 45 g of the desired compound as brown solid.

¹H NMR (400 MHz, CDCl₃) δ: 1.46-1.48 (3H, t), 4.47-4.52 (2H, q), 7.14-7.17 (1H, m), 8.74-8.77 (1H, m), 9.61-9.64 (1H, dd).

m/z=208 (M+H)⁺

### Step B: Preparation of ethyl 2-chloroimidazo[1,2-a]pyrimidine-3-carboxylate

Ethyl 2-hydroxyimidazo[1,2-a]pyrimidine-3-carboxylate (45 g, 217 mmol) was refluxed in phosphorus oxychloride (260 ml, 2.71 mol) for 8 hours. The reaction mixture was cooled and concentrated under vacuo. The reaction mixture was neutralized with saturated sodium bicarbonate solution (100 ml) and extracted with ethyl acetate (3×400 ml). The combined ethyl acetate layer was dried over sodium sulfate and concentrated under vacuo. The obtained crude product was stirred in hexane (100 ml) and separated solid was filtered to give 23.0 g of the desired compound as brown solid.

¹H NMR (400 MHz, CDCl₃) δ: 1.46-1.48 (3H, t), 4.47-4.52 (2H, q), 7.14-7.17 (1H, m), 8.74-8.77 (1H, m), 9.61-9.64 (1H, dd).

m/z=226 (M+H)⁺

### Step C: Preparation of ethyl 2-(4-fluorophenoxy) imidazo[1,2-a]pyrimidine-3-carboxylate

To a stirred solution of Ethyl 2-chloroimidazo[1,2-a]pyrimidine-3-carboxylate 12.5 g (55 mmol) in N, N-dimethylacetamide (60 ml), 4-fluorophenol (7.8 g, 69 mmol) was added and heated at 140° C. for 10 hours. The reaction mixture was concentrated under vacuo and quenched with water (200 ml) and the reaction mixture was extracted with ethyl acetate (3×200 ml). The combined organic layer was dried over sodium sulfate and concentrated under vacuo to give crude product. The crude product was stirred in hexane (100 ml) and separated solid was filtered to give 4.2 g of the desire product as brown solid.

¹H NMR (400 MHz, DMSO-d₆) δ: 1.46-1.48 (3H, t), 4.47-4.52 (2H, q), 7.28-7.33 (5H, m), 8.68-8.70 (1H, dd), 9.57-9.59 (1H, dd).

m/z=302 (M+H)⁺

### Step D: Preparation of 2-(4-fluorophenoxy) imidazo[1,2-a]pyrimidine-3-carboxylic acid

To a stirred solution of ethyl 2-(4-fluorophenoxy) imidazo[1,2-a]pyrimidine-3-carboxylate 0.8 g, 12.6 mmol) in ethanol (25 ml), aqueous solution of sodium hydroxide (1 g, 25.2 mmol) in water (10 ml) was added slowly at 10° C.-12° C. and stirred at room temperature for 4 hours. The reaction mixture was concentrated under vacuo to remove ethanol and water (10 ml) was added. The aqueous layer was washed with ethyl acetate (2×100 ml) and pH of aqueous layer was adjusted to 5 with dilute hydrochloric acid. The separated solid was filtered, washed with water and dried under vacuo to give 2.0 g desired product as brown colored solid.

¹H NMR (400 MHz, DMSO-d₆) δ: 1.46-1.48 (3H, t), 4.47-4.52 (2H, q), 7.28-7.33 (5H, m), 8.68-8.70 (1H, dd), 9.57-9.59 (1H, dd), 13.2 (1H, bs).

m/z=274 (M+H)⁺

### Step E: Preparation of N-(2,4-difluorophenyl)-2-(4-fluorophenoxy)imidazo[1,2-a]pyrimidine-3-carboxamide

To a stirred solution of 2-(4-fluorophenoxy) imidazo[1,2-a]pyrimidine-3-carboxylic acid (0.2 g, 0.7 mmol), N-disopropylethylamine (0.4 ml, 2.2 mmol), 1-hydroxy benzotriazole (0.56 g, 4.2 mmol) and 1-ethyl-3-(3-dimethylamino)propylcarbdiimide hydrochloride (0.28 g, 1.4 mmol) in dichloromethane (20 ml), 2,4-difluoroaniline (0.11 g, 0.7 mmol) was added at 10° C.-15° C. and stirred for 30 minutes. The reaction mixture was stirred at room temperature for 4 hours and concentrated under vacuo. The reaction mixture was diluted with water (20 ml) and the separated solid was filtered, dried under vacuo to give 0.11 g of the desired compound as brown solid.

¹H NMR (400 MHz, DMSO-d₆) δ: 7.13-7.18 (1H, t), 7.32-7.41 (4H, m), 7.49-7.52 (2H, m), 8.00-8.06 (1H, m), 8.71-8.73 (1H, dd), 9.17 (1H, s), 9.72-9.74 (1H, dd).

m/z=338.5 (M+H)⁺

### Step F: Preparation of N-(2,4-difluorophenyl)-2-(4-fluorophenoxy)-N-methylimidazo[1,2-a]pyrimidine-3-carboxamide

To a stirred solution of N-(2,4-difluorophenyl)-2-(4-fluorophenoxyl)imidazo[1,2-a]pyrimidine-3-carboxamide (0.1 g, 0.26 mmol) in tetrahydrofuran (10 ml), sodium hydride (0.03 g, 0.52 mmol) was added portionwise and stirred for 30 minutes. The iodomethane (0.3 g, 7.8 mmol) was added at 10-15° C. and stirred for 4 hours. The reaction mixture was concentrated under vacuo and then cooled water was added and extracted with dichloromethane (3×100 ml). The combined dichloromethane layer dried over sodium sulphate and evaporated under vacuo to yield crude product. The crude product was purified on column chromatography using 30% ethyl acetate in hexane as eluent to give 0.05 g of the desired product as light brown solid.

¹H NMR (400 MHz, CDCl₃) δ: 3.46 (3H, s), 6.73-6.79 (4H, m), 6.97-6.99 (2H, m), 7.06-7.09 (1H, m), 7.17-7.20 (1H, m), 8.57-8.58 (1H, m), 9.28-9.30 (1H, dd).

m/z=399 (M+H)⁺

The following representative compounds of the present invention were prepared in analogues manner by using the synthetic schemes as described above:

TABLE 1

| Comp. | | |
|---|---|---|
| No. | ¹H NMR (400 MHz) | Mass |
| 1 | (CDCl₃) δ: 3.38(3H, s), 6.29(2H, d), 6.81-6.83(2H, d), 7.03- | m/z: 363 |
| | 7.05(1H, t), 7.35-7.37 (5H, m), 8.65-8.67(1H, d), 9.01-9.03(1H, d). | (M+H)⁺ |
| 2 | (CDCl₃) δ: 3.38(3H, s), 6.33(2H, dd), 6.81(1H, d), 6.93(1H, d), 7.04- | m/z: 363 |
| | 7.06(1H, d), 7.35-7.40 (5H, m), 8.66-8.68(1H, d), 9.03-9.05(1H, d). | (M+H)⁺ |
| 3 | (DMSO-d₆ + D₂O) δ: 3.37 (5H, s), 6.59-6.61(2H, d), 6.81- | m/z: 387 |
| | 6.83(2H, d), 7.22-7.25(1H, m), 7.39-7.43(4H, m), 7.52-7.58(1H, t), 8.64- | (M+H)⁺ |
| | 8.65(1H, d), 8.98-9.00(1H, d). | |
| 4 | (CDCl₃) δ: 1.95-2.07(2H, m), 2.60(2H, t), 2.71- | m/z: 403 |
| | 2.75(2H, t), 3.45(3H, s), 6.12-6.23(2H, dd), 6.71(1H, d), 7.02- | (M+H)⁺ |
| | 7.06(1H, m), 7.23-7.3295H, m), 8.65-8.67(1H, m), 8.99-9.02(1H, dd). | |
| 5 | (CDCl₃) δ: 3.41(3H, s), 6.33-6.35(2H, dd), 6.86-6.88(2H, dd), 7.04- | m/z: 397 |
| | 7.07(1H, m), 7.26-7.52(4H, m), 8.67-8.68(1H, m), 9.00-9.02(1H, dd). | (M+H)⁺ |
| 6 | (CDCl₃) δ: 1.94-1.98(2H, m), 2.56(2H, t), 2.69- | m/z: 387 |
| | 2.71(2H, t), 3.41(3H, s), 6.13-6.21(2H, m), 6.71(2H, m), 7.01- | (M+H)⁺ |
| | 7.03(2H, m), 7.37(2H, m), 8.63(1H, m), 8.96-8.98(1H, d). | |
| 8 | (CDCl₃) δ: 3.42(3H, s), 6.34(2H, m), 6.80-6.82(2H, d), 7.04- | m/z: 439 |
| | 7.07(1H, m), 7.39-7.42(3H, m), 7.49-7.53(2H, t), 7.58-7.60(2H, d), 7.66- | (M+H)⁺ |
| | 7.68(2H, d), 8.67-8.69(1H, m), 9.04-9.06(1H, dd). | |
| 9 | (CDCl₃) δ: 1.90-1.94(2H, m), 2.47-2.49(2H, t), 2.70- | m/z: 445 |
| | 2.73(2H, t), 3.43(3H, s), 6.15(2H, m), 6.68(2H, m), 6.99- | (M+H)⁺ |
| | 7.02(2H, m), 7.38-7.41(1H, m), 7.47-7.51(4H, t), 7.57-7.59(2H, d), 7.65- | |
| | 7.67(2H, d), 8.62-8.63(1H, m), 8.96-8.98(1H, d). | |
| 10 | (CDCl₃) δ: 3.40(3H, s), 3.85(3H, s), 3.96(3H, s), 6.31(2H, m), 6.82(2H, m), | m/z: 423 |
| | 6.85-6.87(2H, m), 6.95-6.97(1H, m), 7.02-7.05(1H, m), 8.64-8.66 | (M+H)⁺ |
| | (1H, m), 9.00-9.02(1H, dd). | |
| 12 | (CDCl₃) δ: 3.40(3H, s), 6.34(2H, m), 6.83(2H, d), 7.35- | m/z: 439.5 |
| | 7.38(5H, m), 7.49-7.50(1H, d), 7.53-7.57(2H, t), 7.68(2H, d), 8.92- | (M+H)⁺ |
| | 8.93(1H, d) 9.16-9.17(1H, d). | |
| 13 | (CDCl₃) δ: 1.94-2.0(2H, q), 2.48(2H, t), 2.71(2H, t), 3.40(3H, s), 3 .83(3H, s), | m/z: 429 |
| | 3.9(3H, s), 6.14-6.15(2H, d), 6.69-6.71(1H, d), 6.86-6.88(2H, d), 6.98- | (M+H)⁺ |
| | 7.01(2H, m), 8.60-8.61(1H, m), 8.94-8.96(1H, dd). | |
| 14 | (CDCl₃) δ: 3.44(3H, s), 6.37(2H, m), 6.88-6.90(2H, d), 7.06- | m/z: 431.4 |
| | 7.09(1H, m), 7.23(1H, m), 7.41-7.45(2H, m), 8.69-8.70(1H, d), 9.02- | (M+H)⁺ |
| | 9.04(1H, dd). | |
| 15 | (CDCl₃) δ: 1.92(2H, m), 2.58-2.66(2H, m), 3.37-3.38(2H, m), 5.98- | m/z: 401 |
| | 6.07(2H, m), 6.50-6.68(2H, m), 6.70-6.81(2H, d), 7.05- | (M+H)⁺ |
| | 7.08(1H, m), 8.65-8.66(1H, d), 9.01-9.03(1H, dd), 9.48(1H, s). | |
| 16 | (CDCl₃) δ: 3.38(3H, s), 3.88(3H, s), 6.36(2H, m), 6.85- | m/z: 393 |
| | 6.90(3H, m), 6.99-7.02(1H, m), 7.33-7.35(2H, m), 8.62- | (M+H)⁺ |
| | 8.64(1H, m), 8.97-9.00(1H, dd). | |
| 17 | (CDCl₃) δ: 1.90(2H, m), 2.48(2H, m), 2.66(2H, m), 3.52(3H, s), 6.33- | m/z: 370 |
| | 6.44(2H, m), 6.66(1H, m), 6.96-6.99(1H, m), 7.24-7.26(1H, m), 7.65- | (M+H)⁺ |
| | 7.72(2H, m), 8.60(1H, s), 8.69-8.70(1H, d), 8.74-8.76(1H, dd). | |
| 18 | (CDCl₃) δ: 3.41(3H, s), 6.38(2H, m), 6.59(2H, m), 7.03- | m/z: 381 |
| | 7.05(1H, m), 7.34(4H, m), 8.65-8.67(1H, m), 8.97-9.00(1H, dd). | (M+H)⁺ |
| 19 | (CDCl₃) δ: 3.38(3H, s), 3.43(3H, s), 3.79(3H, s), 5.93-6.10(2H, d), 6.34- | m/z: 423 |
| | 6.35(1H, m), 7.01-7.03(1H, m), 7.34-7.41(4H, m), 8.63- | (M+H)⁺ |
| | 8.64(1H, m), 8.91-9.16(1H, dd). | |
| 20 | (DMSO-d₆) δ: 3.39(3H, s), 6.71-6.73(2H, d), 6.97-6.99(1H, d), 7.25- | m/z: 364 |
| | 7.28(1H, m), 7.41-7.44(1H, m), 7.73-7.75(1H, d), 8.57- | (M+H)⁺ |
| | 8.60(2H, dd), 8.69-8.70(1H, m), 9.09-9.11(1H, dd). | |
| 21 | (CDCl₃) δ: 3.43(3H, s), 6.43-6.45(2H, m), 6.57-6.61(2H, t), 6.94- | m/z: 415, 417 |
| | 6.96(1H, d), 7.07-7.11(2H, m), 7.41-7.42(1H, d), 8.69- | (M+H)⁺ |
| | 8.71(1H, m), 9.18-9.20(1H, dd). | |
| 22 | (CDCl₃) δ: 3.41(3H, s), 6.37(2H, m), 6.59-6.76(2H, t), 6.94- | m/z: 365 |
| | 6.96(1H, d), 7.02-7.07(2H, m), 7.39(2H, m), 8.65-8.66(1H, m), 8.98- | (M+H)⁺ |
| | 9.00(1H, dd). | |
| 23 | (CDCl₃) δ: 3.32(3H, s), 6.30-6.32(2H, d), 6.86-6.88(2H, d), 6.95- | m/z: 431 |
| | 6.96(1H, d), 7.10-7.13(1H, m), 7.43-7.47(1H, t), 7.50-7.53(1H, t), 7.66- | (M+H)⁺ |
| | 7.68(1H, d), 8.70-8.71(1H, m), 9.18-9.20(1H, dd). | |
| 24 | (CDCl₃) δ: 2.67 (6H, s), 3.48(3H, s), 6.59-6.61(2H, d), 7.08- | m/z: 470 |
| | 7.11(1H, m), 7.32-7.35 (6H, m), 8.70-8.72(1H, m), 9.06-9.09(1H, dd). | (M+H)⁺ |
| 25 | (CDCl₃) 6 :3.24(3H, s), 3.34(3H, s), 3.64(3H, s), 5.91- | m/z: 423 |
| | 5.92(1H, d), 5.99(1H, s), 6.11-6.13(1H, d), 6.94-6.97(1H, m), 7.33- | (M+H)⁺ |
| | 7.35(2H, d), 7.46-7.48(2H, d), 8.58(1H, m), 8.85-8.87(1H, dd). | |
| 26 | (CDCl₃) δ: 3.38(3H, s), 6.34-6.55(4H, m), 7.01-7.04(1H, m), 7.35- | m/z: 347 |
| | 7.40 (5H, m), 8.65(1H, m), 8.98-9.00(1H, dd). | (M+H)⁺ |
| 27 | (DMSO-d₆) δ: 3.36(3H, s), 6.04-(1H, m), 6.16-6.27(2H, dd), 7.12- | m/z: 395 |
| | 7.15(1H, m), 7.52-7.54(2H, d), 7.67-7.69(2H, d), 8.58- | (M+H)⁺ |
| | 8.59(1H, m), 8.78(1H, m), 8.91-8.96(2H, dd). | |
| 28 | (CDCl₃) δ: 3.40(3H, s), 6.02(2H, s), 6.46(2H, m), 6.65(2H, m), 6.80- | m/z: 391 |
| | 6.82(1H, d), 6.90-6.95(2H, m), 6.99-7.02(1H, m), 8.62- | (M+H)⁺ |
| | 8.63(1H, m), 8.95-8.97(1H, dd). | |
| 29 | (CDCl₃) δ: 3.40(3H, s), 6.02(2H, s), 6.42(2H, m), 6.79- | m/z: 407 |
| | 6.81(1H, d), 6.86(1H, m), 6.90-6.92(3H, m), 7.00- | (M+H)⁺ |
| | 7.03(1H, m), 8.64(1H, m), 8.98-9.00(1H, dd). | |
| 30 | (CDCl₃) δ: 3.35(3H, s), 6.35(2H, m), 6.72- | m/z: 379 |
| | 6.74(2H, d), 6.85(2H, m), 7.04-7.10(3H, m), 8.66- | (M+H)⁺ |
| | 8.67(1H, m), 8.85(1H, bs), 9.02-9.03(1H, dd). | |
| 31 | (DMSO-d₆) δ: 3.38(3H, s), 6.35-6.55(2H, d), 6.93-6.95(2H, d), 7.27- | mh: 431 |
| | 7.30(1H, m), 7.52(1H, s), 7.65-7.69(2H, m), 7.78-7.80(1H, d), 8.71- | (M+H)⁺ |
| | 8.72(1H, m), 9.10-9.12(1H, dd). | |
| 33 | (CDCl₃) δ: 3.44(3H, s), 6.32(2H, m), 6.55(2H, m), 7.08- | m/z: 415 |
| | 7.11(1H, m), 7.47-7.51(1H, m), 7.61-7.67(3H, m), 8.71- | (M+H)⁺ |
| | 8.72(1H, m), 9.06-9.08(1H, dd). | |
| 34 | (DMSO-d₆) δ: 3.63(3H, s), 3.75(3H, s), 6.53- | m/z: 393 |
| | 6.54(1H, d), 6.60(1H, m), 7.29-7.31(1H, q), 7.38- | (M+H)⁺ |
| | 7.43(2H, t), 7.91(3H, m), 8.74(1H, m), 8.85(1H, s), 9.48-9.49(1H, d). | |
| 35 | (DMSO-d₆) δ: 3.46(3H, s), 6.39(2H, m), 6.60(2H, m). 7.10(1H, m), 7.28 | m/z: 372 |
| | (1H, s), 7.49-7.50(1H, t), 7.68-7.74(2H, m), 8.72(1H, m), | (M+H)⁺ |
| | 9.02-9.04(1H, dd). | |
| 36 | (CDCl₃) δ: 3.46(3H, s), 6.32-6.34(2H, d), 6.85-6.87(2H, d). 7.09- | m/z: 388 |
| | 7.12(1H, m), 7.45-7.49(1H, t), 7.62-7.64(1H, d), 7.67-7.68(2H, d), 8.71- | (M+H)⁺ |
| | 8.73(1H, m), 9.04-9.07(1H, dd). | |
| 37 | (CDCl₃) δ: 2.75 (6H, s), 3.43(3H, s), 6.31(2H, d), 6.84- | m/z: 470 |
| | 6.86(2H, d). 7.09-7.12(1H, m), 7.54-7.56(2H, d), 7.74- | (M+H)⁺ |
| | 7.76(2H, dd), 8.71-8.73(1H, m), 9.04-9.07(1H, dd). | |
| 38 | (CDCl₃) δ: 2.75 (6H, s), 3.44(3H, s), 6.36(2H, m), 6.60(2H, m). 7.10- | m/z: 454 |
| | 7.12(1H, m), 7.59-7.60(2H, m), 7.77-7.79(2H, d), 8.71(1H, m), 9.02- | (M+H)⁺ |
| | 9.04(1H, dd). | |
| 39 | (DMSO-d₆) δ: 7.14(1H, m), 7.28-7.35(4H, m), 7.75(1H, m), 7.95 | m/z: 369 |
| | (2H, m), 8.74(1H, s), 9.24(1H, dd), 10.04(1H, s). | (M+H)⁺ |
| 40 | (CDCl₃) δ: 3.41(3H, s), 6.27(1H, d), 6.40(2H, m). 7.03-7.05 | m/z: 383 |
| | (1H, m), 7.10(2H, m), 7.42(2H, m), 8.67(1H, m), 8.95(1H, dd). | (M+H)⁺ |
| 41 | (CDCl₃) δ: 3.43(3H, s), 3.98(3H, s), 6.46(2H, m), 6.64(2H, m), 6.94- | m/z: 395 |
| | 6.98(1H, t), 7.03-7.05(1H, m), 7.22-7.28(2H, m), 8.66-8.67(1H, d), 8.97- | (M+H)⁺ |
| | 8.98(1H, d). | |
| 42 | (CDCl₃) δ: 3.42(3H, s), 6.44(2H, m), 6.65(2H, m), 6.91- | m/z: 381 |
| | 6.99(1H, t), 7.01-7.05(1H, m), 7.28-7.34(2H, m), 8.66-8.67(1H, d), 8.99- | (M+H)⁺ |
| | 9.00(1H, d). | |
| 43 | (CDCl₃) δ: 3.43(3H, s), 3.93(3H, s), 6.35-6.43(3H, m), 6.99-7.05 | m/z: 413 |
| | (3H, m), 7.14-7.22(2H, m), 8.66-8.67(1H, d), 8.99-9.00(1H, d). | (M+H)⁺ |
| 44 | (CDCl₃) δ: 3.43(3H, s), 3.98(3H, s), 6.37-6.43(2H, m), 6.90- | m/z: 411 |
| | 6.97(3H, m), 7.04-7.06(1H, m), 7.19-7.23(2H, d), 8.66- | (M+H)⁺ |
| | 8.67(1H, m), 8.99-9.01(1H, dd). | |
| 45 | (CDCl₃) δ: 3.42(3H, s), 3.99(3H, s), 6.33(1H, bs), 6.55(1H, bs), | m/z: 429 |
| | 6.70-6.74(1H, t), 6.96-6.98(1H, m), 7.06-7.08(1H, m), 7.16- | (M+H)⁺ |
| | 7.19(2H, m), 8.68-8.69(1H, m), 9.03-9.05(1H, dd). | |
| 47 | (CDCl₃) δ: 3.52(3H, s), 6.72-6.74(2H, d), 7.06-7.12(3H, m), 7.14- | m/z: 379.5 |
| | 7.19(3H, m), 7.27-7.31(2H, m), 8.56-8.58(1H, m), 9.34-9.36(1H, dd). | (M+H)⁺ |
| 48 | (CDCl₃) δ: 3.52(3H, s), 6.66-6.68(2H, d), 7.07-7.10(3H, m), 7.17- | m/z: 413, 415 |
| | 7.19(2H, d), 7.24-7.27(2H, m), 8.58-8.59(1H, m), 9.34-9.36(1H, dd). | (M+H)⁺ |
| 49 | (CDCl₃) δ: 3.53(3H, s), 3.80(3H, s), 6.63-6.65(2H, d), 6.80- | m/z: 409 |
| | 6.86(2H, d), 7.05-7.12(3H, m), 7.20-7.22(2H, d), 8.54- | (M+H)⁺ |
| | 8.56(1H, m), 9.33-9.36(1H, dd). | |
| 50 | (CDCl₃) δ: 3.51(3H, s), 6.63-6.67(2H, d), 6.93-6.97(2H, t), 7.05- | m/z: 397.5 |
| | 7.09(3H, m), 7.16-7.18(2H, d), 8.55(1H, m), 9.32-9.33(1H, dd). | (M+H)⁺ |
| 51 | (CDCl₃) δ: 3.52(3H, s), 6.69-6.72(2H, d), 6.90-6.94(2H, t), 7.07- | m/z: 397.5 |
| | 7.09(1H, m), 7.11-7.14(2H, m), 7.23-7.28(2H, d), 8.57- | (M+H)⁺ |
| | 8.59(1H, m), 9.31-9.35(1H, dd). | |
| 52 | (CDCl₃) δ: 3.36(3H, s), 3.71(3H, s), 6.54-6.69(3H, m), 6.92- | m/z: 393 |
| | 6.95(2H, t), 7.04-7.06(3H, m), 8.54(1H, d), 9.30-9.32(1H, d). | (M+H)⁺ |
| 53 | (CDCl₃) δ: 3.43(3H, s), 6.41(2H, d), 6.81(1H, s), 6.92-6.98(3H, | m/z: 397 |
| | m), 7.04-7.08(3H, m), 8.69(1H, s), 9.03-9.04(1H, dd). | (M+H)⁺ |
| 54 | (CDCl₃) δ: 3.46(3H, s), 6.33(2H, d), 6.86-6.87(2H, d), 7.11(1H, | mh :388 |
| | m), 7.54(2H, m), 7.66(2H, d), 8.73(1H, s), 9.04(1H, s). | (M+H)⁺ |
| 55 | (CDCl₃) δ: 3.46(3H, s), 6.38(2H, d), 6.61(2H, d), 7.11(1H, m), | m/z: 372 |
| | 7.58(2H, m), 7.68(2H, d), 8.72(1H, d), 9.04(1H, dd). | (M+H)⁺ |
| 56 | (CDCl₃) δ: 3.43(3H, s), 6.28-6.42(2H, dd), 6.70(1H, d), 7.11(1H, m), | m/z: 406 |
| | 7.53(2H, d), 7.71-7.79(2H, d), 8.74(1H, s), 9.04(1H, dd). | (M+H)⁺ |
| 57 | (CDCl₃) δ: 4.92-5.02(2H, s), 6.17-6.18(2H, dd), 6.79-6.81 | m/z: 475 |
| | (2H, dd), 7.01-7.07(4H, m), 7.27-7.29(2H, m), 7.35(2H, d), | (M+H)⁺ |
| | 8.68(1H, d), 9.01-9.02(1H, dd). | |
| 58 | (CDCl₃) δ: 3.52(3H, s), 6.84-6.87(2H, m), 7.07-7.16(2H, m), | m/z: 449 |
| | 7.33-7.34(3H, m), 8.55(1H, d), 9.31-9.32(1H, dd). | (M+H)⁺ & |
| | | 451 |
| | | (M+2H)⁺ |
| 59 | (CDCl₃) δ: 3.59(3H, s), 7.02-7.07(3H, m), 7.12-7.13(1H, m), | m/z: 431 |
| | 7.25-7.32(2H, m), 7.34(2H, m), 8.55(1H, d), 9.34-9.35(1H, dd). | (M+H)⁺ & |
| | | 433 |
| | | (M+2H)⁺ |
| 60 | (CDCl₃) δ: 3.55(3H, s), 6.67-6.68(1H, d), 6.80(1H, m), 6.86- | m/z: 415 |
| | 6.90(1H, d), 7.15(1H, m), 7.24(2H, m), 7.29(2H, m), 8.58(1H, d), | (M+H)⁺ |
| | 9.37-9.38(1H, dd). | |
| 61 | (CDCl₃) δ: 3.37(3H, s), 6.56-6.74(4H, m), 6.91-6.92(1H, d), | m/z: 387 |
| | 6.99-7.01(1H, m), 7.11-7.12(1H, d), 8.62-8.63(1H, d), 8.80-8.82 | (M+H)⁺ |
| | (1H, dd). | |
| 62 | (CDCl₃) δ: 3.38(3H, s), 6.38-6.47(2H, d), 6.79-6.84(1H, d), | m/z: 404 |
| | 6.84-7.00(4H, m), 8.64-8.65(1H, d), 8.83-8.84(1H, dd). | (M+H)⁺ |
| 63 | (CDCl₃) δ: 3.43(3H, s), 6.28-6.29(2H, d), 6.80-6.82(2H, d), | m/z: 431 |
| | 7.09(1H, s), 7.48-7.50(2H, d), 7.60-7.62(2H, d), 8.71(1H, s), | (M+H)⁺ |
| | 9.05-9.07(1H, d). | |
| 64 | (CDCl₃) δ: 3.44(3H, s), 6.33-6.55(4H, m), 7.07-7.10(1H, m), | m/z: 415 |
| | 7.54-7.63(4H, m), 8.70-8.71(1H, m), 9.03-9.05(1H, m). | (M+H)⁺ |
| 65 | (DMSO-d₆) δ: 0.96-1.22 (6H, s), 4.97(1H, s), 6.52-6.62 | m/z: 409 |
| | (2H, s), 7.05(2H, s), 7.23-7.26(1H, m), 7.37- | (M+H)⁺ |
| | 7.39(2H, m), 7.58(2H, m), 8.65-8.66(1H, d), 9.04-9.06(1H, dd). | |
| 66 | (CDCl₃) δ: 7.14-7.20(3H, m), 7.35-7.54(4H, m), 7.60-7.73 | m/z: 383 |
| | (2H, d), 8.65-8.67(2H, m), 9.91-9.93(1H, dd). | (M+H)⁺ |
| 67 | (DMSO-d₆) δ:7.18-7.22(2H, m), 7.25-7.28(1H, m), 7.59(2H, s), | m/z: 401 |
| | 7.68-7.72(1H, m), 7.77-7.79(1H, m), 8.14-8.18(2H, m), 8.74-8.75) | (M+H)⁺ |
| | 1H, dd), 9.18-9.20(1H, dd). | |
| 68 | (CDCl₃) δ: 0.48(2H, d), 0.79-0.81(2H, d), 2.91(1H, m), 6.72-6.76 | m/z: 390 |
| | (2H, m), 7.01-7.04(1H, m), 7.60-7.62(2H, m), 7.48- | (M+H)⁺ |
| | 7.59(2H, m), 8.66-8.68(1H, m), 8.92-8.94(1H, m) | |
| 69 | (DMSO-d₆) δ: 0.44-0.56(2H, d), 0.76-0.86(2H, d), 3.03(1H, m), | m/z: 441 |
| | 6.80(2H, s), 6.85-6.89(2H, t), 7.24-7.27(1H, m), 7.40-7.77 | (M+H)⁺ |
| | (2H, m), 7.83-7.90(2H, m), 8.69-8.70(1H, dd), 9.06-9.08(1H, dd). | |
| 70 | (DMSO-d₆) δ: 0.35-0.54(4H, m), 3.01(1H, m), 6.76(2H, m), | m/z: 457 |
| | 7.07(2H, m), 7.27-7.33(1H, m), 7.72-7.84(4H, m), 8.70 | (M+H)⁺ |
| | (1H, d), 9.06-9.07(1H, dd). | |
| 131 | (CDCl₃) δ: 3.34-3.39(4H, m), 3.51(3H, s), 3.72-3.86(4H, m), | m/z: 356 |
| | 6.89-6.99 (SH, m), 8.48-8.49(1H, d), 8.97-9.00(1H, d). | (M+H)⁺ |
| 132 | (CDCl₃) δ: 3.53(3H, s), 6.43-6.55(2H, m), 6.85-6.86(1H, m), | m/z: 397 |
| | 7.07-7.28 (6H, m), 8.59-8.61(1H, dd), 9.34-9.37(1H, dd). | (M+H)⁺ |
| 133 | (CDCl₃) δ: 3.46(3H, s), 3.67(3H.s), 6.30-6.39(4H, m), 7.01- | m/z: 377 |
| | 7.08(3H, m), 7.42(2H, s), 8.64-8.65(1H, dd), 8.94-8.96(1H, dd). | (M+H)⁺ |
| 134 | (CDCl₃) 6 :1.27-1.32(3H, t), 3.51(3H, s), 4.33-4.36(2H.q), 6.44- | m/z: 419 |
| | 6.46(2H, d), 6.89-7.08(3H, m), 7.28-7.31(2H, m), 7.58- | (M+H)⁺ |
| | 7.60(2H, m), 8.69(1H, dd), 9.05-9.06(1H, dd). | |
| 135 | (CDCl₃) δ: 3.41(3H, s), 3.78(3H.s), 6.35-6.37(2H, d), 6.58- | m/z: 411 |
| | 6.62(2H, m), 6.83-6.85(2H, d), 7.01-7.03(1H, m), 7.10- | (M+H)⁺ |
| | 7.12(1H, m), 8.63-8.64(1H, dd), 9.03-9.05(1H, dd). | |
| 136 | (CDCl₃) δ: 0.50(2H, d), 0.82-0.83(2H, d), 2.89-2.90(1H, m), | m/z: 407 |
| | 6.54-6.57(2H, d), 7.00-7.02(2H, d), 7.05-7.07(1H, m), 7.12-7.16 | (M+H)⁺ |
| | (2H, m), 7.52-7.56(2H, m), 8.67-8.69(1H, m), 8.94-8.96(1H, dd). | |
| 137 | (DMSO-d₆) δ:3.36(3H, s), 6.74-6.76(2H, d), 6.98-7.00 | m/z: 381 |
| | (2H, d), 7.22-7.28(3H, m), 7.41-7.44(2H, m), 8.68-8.70 | (M+H)⁺ |
| | (1H, m), 9.06-9.08(1H, dd). | |
| 138 | (DMSO-d₆) δ: 3.39(3H, s), 6.78-6.80(2H, d), 6.99-7.03 | m/z: 381 |
| | (2H, d), 7.27-7.31(2H, m), 7.36-7.39(1H, m), 7.42-7.46(2H, m), | (M+H)⁺ |
| | 8.78-8.79(1H, m), 9.14-9.16(1H, dd). | |
| 139 | (DMSO-d₆) δ: 2.30(3H, s), 3.39(3H, s), 6.78-6.80(2H, d), 7.01-7.03 | m/z: 381 |
| | (2H, d), 7.12-7.14(2H, d), 7.27-7.31(2H, m), 7.34-7.37 | (M+H)⁺ |
| | (2H, m), 7.44-7.50(3H, m), 8.76-8.77(1H, m), 9.13-9.15(1H, dd). | |
| 140 | (DMSO-d₆) δ: 3.38(3H, s), 3.73(3H, s), 6.78-6.80(2H, d), 7.01-7.03 | m/z: 381 |
| | (2H, d), 7.27-7.31(2H, m), 7.35-7.40(1H, m), 8.76- | (M+H)⁺ |
| | 8.78(1H, m), 9.14-9.16(1H, dd). | |
| 141 | (DMSO-d₆) δ: 2.80-2.81(3H, d), 7.09-7.12(1H, q), 7.32-7.40 | m/z: 271 |
| | (2H, m), 7.87-7.91(2H, q), 8.28-8.29(1H, m), 8.67-8.70 | (M+H)⁺ |
| | (1H, m), 9.12-9.14(1H, dd). | |
| 142 | (CDCl₃) δ: 1.16-1.39 (6H, m), 1.71-1.74(2H, m), 1.80-1.83 | m/z: 429 |
| | (2H, m), 2.28-2.31(1H, m), 3.44(3H, s), 6.27(2H, m), 6.70 | (M+H)⁺ |
| | (2H, m), 6.98-7.05(3H, m), 7.28-7.31(2H, m), 8.65-8.66 | |
| | (1H, m), 9.01-9.03(1H, dd). | |
| 143 | (CDCl₃) δ: 3.00(4H, m), 3.43(3H, s), 3.80-3.82(4H, m), | m/z: 429 |
| | 6.28(2H, m), 6.37(2H, m), 7.01-7.06(3H, m), 7.43(2H, m), | (M+H)⁺ |
| | 8.63-8.65(1H, m), 8.95-8.97(1H, dd). | |
| 165 | (DMSO-d₆) δ: 2.29(3H, s), 3.42(3H, s), 6.70-6.73(2H, dd), | m/z: 397.2 |
| | 7.11-7.15(4H, m), 7.22-7.24(2H, d), 7.29-7.32(1H, m), | (M+H)⁺ |
| | 7.34-7.36(2H, d), 7.46-7.48(2H, d), 8.61-8.62(1H, m), | |
| | 9.26-9.27(1H, dd). | |
| 166 | (DMSO-d₆) δ: 3.42(3H, s), 6.11(1H, bs), 6.70-6.73(2H, dd), | m/z: 397.2 |
| | 7.11-7.13(2H, t), 7.22-7.25(2H, d), 7.29-7.37 (6H, m), | (M+H)⁺ |
| | 7.59-7.61(2H, dd), 8.61-8.62(1H, m), 9.26-9.28(1H, dd). | |

### Combination Therapy

Compounds of the present invention may be administered in combination with other drugs that are used in the treatment/prevention/suppression or amelioration of the diseases or conditions for which compounds of Formula (I) are useful. Such other drugs may be administered contemporaneously or sequentially with a compound of Formula (I). When a compound of Formula (I) is used contemporaneously with one or more other drugs, a pharmaceutical composition containing such other drugs in addition to the compound of Formula (I) is preferred. Accordingly, the pharmaceutical compositions of the present invention include those that also contain one or more other active ingredients, in addition to a compound of Formula (I).

### Pharmaceutical Compositions

In another embodiment of the invention, there is provided a pharmaceutical composition comprising a therapeutically effective amount of one or more of a compound of formula (I). While it is possible to administer therapeutically effective quantity of compounds of formula (I) either individually or in combination, directly without any formulation, it is common practice to administer the compounds in the form of pharmaceutical dosage forms comprising pharmaceutically acceptable excipient(s) and at least one active ingredient. These dosage forms may be administered by a variety of routes including oral, topical, transdermal, subcutaneous, intramuscular, intravenous, intreperitoneal, intranasal, pulmonary etc.

Oral compositions may be in the form of solid or liquid dosage form. Solid dosage form may comprise pellets, pouches, sachets or discrete units such as tablets, multi-particulate units, capsules (soft & hard gelatin) etc. Liquid dosage forms may be in the form of elixirs, suspensions, emulsions, solutions, syrups etc. Composition intended for oral use may be prepared according to any method known in the art for the manufacture of the composition and such pharmaceutical compositions may contain in addition to active ingredients, excipients such as diluents, disintegrating agents, binders, solubilizers, lubricants, glidants, surfactants, suspending agents, emulsifiers, chelating agents, stabilizers, flavours, sweeteners, colours etc. Some example of suitable excipients include lactose, cellulose and its derivatives such as microcrystalline cellulose, methylcellulose, hydroxy propyl methyl cellulose & ethylcellylose, dicalcium phosphate, mannitol, starch, gelatin, polyvinyl pyrolidone, various gums like acacia, tragacanth, xanthan, alginates & its derivatives, sorbitol, dextrose, xylitol, magnesium Stearate, talc, colloidal silicon dioxide, mineral oil, glyceryl mono stearate, glyceryl behenate, sodium starch glycolate, cross povidone, crosslinked carboxymethylcellulose, various emulsifiers such as polyethylene glycol, sorbitol, fatty acid esters, polyethylene glycol alkylethers, sugar esters, polyoxyethylene polyoxypropyl block copolymers, polyethoxylated fatty acid monoesters, diesters and mixtures thereof.

Sterile compositions for injection can be formulated according to conventional pharmaceutical practice by dissolving or suspending the active substance in a vehicle such as water for injection, N-Methyl-2-Pyrrolidone, propylene glycol and other glycols, alcohols, a naturally occurring vegetable oil like sesame oil, coconut oil, peanut oil, cotton seed oil or a synthetic fatty vehicle like ethyl oleate or the like. Buffers, anti-oxidants, preservatives, complexing agents like cellulose derivatives, peptides, polypeptides and cyclodextrins and the like can be incorporated as required.

The dosage form can have a slow, delayed or controlled release of active ingredients in addition to immediate release dosage forms.

The amount of active ingredient which is required to achieve a therapeutic effect will, of course, vary with the particular compound, the route of administration, the subject under treatment, and the particular disorder or disease being treated. The compounds of the invention may be administered orally or parenteraly at a dose of from 0.001 to 1500 mg/kg per day, preferably from 0.01 to 1500 mg/kg per day, more preferably from 0.1 to 1500 mg/kg per day, most preferably from 0.1 to 500 mg/kg per day. The dose range for adult humans is generally from 5 mg to 35 g per day and preferably 5 mg to 2 g per day.

Dosage forms of presentation provided in discrete units may conveniently contain an amount of compound of the invention which is effective at such dosage or as a multiple of the same, for example units containing 5 mg to 1500 mg.

### Biological Testing:

### In Vitro Screening:

(A) cAMP Responsive Element (CRE)-Reporter Assay:

CHO cells (ATCC) were transfected with human TGR5 (oriGene) and CRE-luciferase reporter vector. Transfected cells were treated with vehicle control or test compounds (Conc 10 μM) for five hours and then lysed. Cell lysates were monitored for luciferase acivity. Increase in luciferase activity is considered as a result of TGR5 activation. Results were expressed as fold induction as compared to vehicle control.

### Results:

Results are summarized in Table 2 where, + indicates 1.5-2 fold while ++, +++, ++++ indicate 2-3 fold, 3-4 fold, and >4 fold induction respectively relative to vehicle control when tested at 10 μM concentration.

TABLE 2

| Comp. | |
|---|---|
| No. | Fold Induction |
| 1 | ++++ |
| 2 | +++ |
| 3 | + |
| 4 | +++ |
| 5 | ++++ |
| 6 | +++ |
| 7 | ++++ |
| 8 | +++ |
| 9 | ++ |
| 10 | +++ |
| 11 | + |
| 12 | ++ |
| 13 | +++ |
| 14 | +++ |
| 15 | ++ |
| 16 | ++ |
| 17 | ++ |
| 18 | ++ |
| 19 | ++ |
| 20 | ++ |
| 21 | +++ |
| 22 | +++ |
| 23 | +++ |
| 24 | ++ |
| 25 | ++ |
| 26 | ++ |
| 27 | + |
| 28 | ++ |
| 29 | +++ |
| 30 | +++ |
| 31 | +++ |
| 32 | + |
| 33 | ++++ |
| 34 | + |
| 35 | ++ |
| 36 | ++ |
| 37 | + |
| 38 | + |
| 39 | + |
| 40 | ++ |
| 41 | ++ |
| 42 | ++ |
| 43 | + |
| 44 | +++ |
| 45 | ++++ |
| 46 | ++ |
| 47 | +++ |
| 48 | ++ |
| 49 | +++ |
| 50 | ++++ |
| 51 | ++ |
| 52 | +++ |
| 136 | +++ |
| 65 | +++ |

(B) cAMP Measurement Assay:

CHO cells (ATCC) were transfected with human TGR5 vector (oriGene). Transfected cells were treated with vehicle control or test compounds for one hour and then lysed. Levels of cAMP were measured in cell lysates employing Alphascreen cAMP assay kit (Perkin Elmer) and results were expressed EC50 values which are summarized in Table 3.

TABLE 3

| Comp. No. | EC₅₀ nM |
|---|---|
| 1 | 35 |
| 7 | 90 |
| 33 | 155 |
| 50 | 354 |

### (C) Measurement of Glucagon Like Peptide-1 (GLP-1):

Human enteroendocrine cell-line (NCI-H716) were incubated with vehicle or test compounds for one hour. At the end of incubation period, levels of secreted GLP-1 in culture medium were measured by GLP-1 ELISA kit (Millipore). Results are summarized as fold increase in GLP secretion with respect to vehicle control in Table 4.

TABLE 4

| | Fold increase in |
|---|---|
| Comp. No. | GLP Release |
| 7 | 3.72 |
| 22 | 2.13 |
| 28 | 2.03 |
| 30 | 1.69 |
| 33 | 1.83 |
| 50 | 1.74 |

### In Vivo Studies:

i) Evaluation of single dose efficacy of test compounds on glucose tolerance in diabetic hamster

ii) Evaluation of efficacy of test compounds on repeated dosing in diabetic hamster and DIO mice.

### i) Evaluation of Single Dose Efficacy of Test Compounds on Glucose Tolerance:

TGR5 receptor activation results in GLP-1 secretion which, in turn, stimulates insulin release from pancreatic B cells & hence effectively controls the post prandial glucose excursions. Thus efficacy can be assessed through the effect of test compounds on lowering of plasma glucose during OGTT through stimulating glucose stimulated insulin secretion. Hence the effect of test compounds on glucose lowering during OGTT was assessed in diabetic hamster model.

The potential of test compounds in lowering of Plasma Glucose was evaluated in Oral glucose tolerance test (OGTT) in Diabetic Hamster Model, where diabetes has been induced by administration of low dose of Streptozotocin (STZ) to the High fat diet (HFD) fed glucose intolerant animals. The diabetic hamsters show impaired glucose stimulated insulin secretion and higher plasma glucose excursions than the normal animals, which remains elevated beyond 2 hrs post glucose load. Thus the animal model can be used for evaluating the potential of test compounds to lower plasma glucose through stimulating glucose stimulated insulin secretion. On the day of study post 6 hrs of fasting, test compounds or vehicle was administered orally at dose volume of 2 ml/kg to the hamsters of the respective treatment groups. Subsequent to dosing, a pre-glucose load blood sample was taken. A glucose load of 40% solution at 5 ml/Kg dose volume was administered orally. Blood samples through retroorbital plexus were taken at 15, 30 60 & 120 min. post glucose load and plasma was separated for glucose measurement. Post glucose load percentage change in plasma glucose and AUC of % change glucose by the treatment was assessed.

TABLE 5
Effect of compounds on glucose excursion during
OGTT in Diabetic Hamster

| | | | Decrease in AUC of |
|---|---|---|---|
| | | | glucose % change w.r.t. |
| No. | Treatment | Dose | vehicle control group |
| 1 | Compound no. 1 | 36 mg/kg | 33% |
| 2 | Compound no. 7 | 38 mg/kg | 39% |
| 3 | Compound no. 33 | 41 mg/kg | 20% |
| 4 | Compound no. 28 | 39 mg/kg | 5% |
| 5 | Compound no. 22 | 36 mg/kg | 20% |
| 6 | Compound no. 50 | 40 mg/kg | 10% |
| 7 | Compound no. 36 | 38 mg/kg | 8% |
| 8 | Compound no. 65 | 41 mg/kg | 10% |

ii) Evaluation of Efficacy of Test Compounds on Repeated Dosing in Diabetic Hamster and DIO Mice.

TGR5 plays a role in regulating energy expenditure by increasing basal metabolism by increasing cellular conversion of T4 to T3 through TGR5-dependent induction of deiodinase 2 (Dio2). Dio2 is a gene whose protein product is the enzyme 2-iodothyronine deiodinase or D2. D2 actually converts locally available thyroxine (T4) to tri-iodothyronine (T3), resulting in increased energy expenditure without leading to changes in circulating thyroid hormone levels. TGR5 also found to be expressed in liver sinusoidal endothelial cells as well as in Kupffer cells. TGR5 activation induces glucose stimulated insulin release through increasing incretin secretion, increases energy expenditure, inhibit cytokine production, induces body weight reduction, improve insulin resistance and glycemic profile and reduces hepatic steatosis. Thus TGR5 activation has potential to improve various cardiometabolic risk factors associated with obesity and type 2 diabetes. Hence, the efficacy of test compounds was evaluated in diabetic hamster and mouse model with these metabolic derangements.

### a) Efficacy Study in Diabetic Hamster

Diabetic hamsters were randomized to two treatment groups viz. vehicle treated and test compound treated group. Then the animals were treated with compound 7 of present invention or vehicle for 2 weeks to assess the efficacy potential of the compound. Effect of treatment on glucose excursion and insulin secretion during OGTT, change in body weight & fasting and random plasma triglycerides (TG) was evaluated during the treatment period. The effect of repeated administration of compound on energy expenditure was evaluated through monitoring oxygen consumption (VO₂) over 21 hrs period by indirect calorimetry (Oxymax System, Columbus Instruments). HOMA-IR an index of insulin resistance was estimated using fasting glucose and insulin levels estimated during OGTT.

Similarly, the study was conducted using compound no 50 of the present invention for the duration of four week period in Diabetic hamsters.

In diabetic hamster, treatment with compound no. 7 increased energy expenditure, reduced body weight, reduced glucose excursion and improved insulin secretion in response to oral glucose load, improved insulin resistance as evident by decrease in HOMA-IR and decreased plasma TG levels (Table 6). The treatment with compound no. 7 also shown improvement in HDL:LDL ratio by 24% (increase in HDL by 7% and decrease in non HDL & LDL by 24% & 14% respectively). Similarly, compound no. 50 increased energy expenditure, reduced body weight, reduced glucose excursion and decreased plasma TG levels in diabetic hamsters (Table 7). The treatment with compound no. 50 also shown improvement in HDL:LDL ratio by 16%, and decrease in non HDL & LDL by 24% & 16% respectively

### b) Efficacy Study in DIO Mice

Male C57B1/6J mice were made insulin resistant by feeding on High fat diet (45.5% Kcal from Fat, Research Diet) from the age of 6-8 week onwards. After being on High fat diet for 6-8 weeks the animals with similar body weights & fasting plasma glucose were further randomized into treatment groups. Then the animals were treated with compounds 7 to assess the efficacy potential during treatment duration. Effect of treatment on glucose excursion and insulin secretion during OGTT, Fasting Plasma Glucose & Insulin, lipid profile and body weight were evaluated. The compound for enhancing energy expenditure was evaluated by monitoring oxygen consumption (VO₂) and carbon dioxide release (VCO₂) over 24 hrs period by indirect calorimetry (Oxymax System, Columbus Instruments).

Similarly, the study was conducted in DIO mice with compound no 50 of the present invention.

In DIO mice treatment with compound no. 7 increased energy expenditure, reduced body weight, reduced glucose excursion and improved insulin secretion in response to oral glucose load, improved insulin resistance as evident by decrease in HOMA-IR and decreased plasma TG levels (Table 8). Similarly, treatment with compound no. 50 in DIO mice increased energy expenditure, reduced body weight, reduced glucose excursion and improved insulin secretion in response to oral glucose load, improved insulin resistance as evident by decrease in HOMA-IR (Table 9).

TABLE 6
Effect of compound no. 7 on various metabolic parameters on repeated administration in diabetic hamster

| | % Change | Oxygen | AUC_glucose | AUC_Insulin | | Fasting | Random |
|---|---|---|---|---|---|---|---|
| Treatment | in body wt | consumption, | % change | % change | | plasma | plasma |
| (dose) | fro basal | ml/kg/h | during OGTT | during OGTT | HOMA-IR | TG (mg/dl) | T [symbol] (mg/dl) |
| Vehicle control | −0.9 ± 0.9 | 1254.50 ± 33.1 | 15364.52 ± 1363.2 | 1267.0 ± 875.9 | 22.96 ± 3.2 | 329.37 ± 47.4 | 550.70 ± 65 |
| Compound no. 7, | −5.2 ± 1.6 | 1353.06 ± 38.3 | 12714.43 ± 912 | 2280.6 ± 428.8 | 15.6 ± 2.1 | 188.35 ± 3.7 | 344.38 ± 90.3 |
| (20 mg/kg, po, bid) | | | | | | | |

[symbol]

indicates data missing or illegible when filed

TABLE 7
Effect of compound no. 50 on various metabolic parameters
on repeated administrationin diabetic hamster

| | % Change | | AUC_glucose | Fasting |
|---|---|---|---|---|
| | in body | Oxygen | % change | plasma |
| Treatment | wt from | consumption, | during | TG |
| (dose) | basal | ml/kg/h | OGTT | (mg/dl) |
| Vehicle | 1.32 ± | 1446.25 ± | 9604.5 ± | 132.9 ± |
| control | 0.59 | 28.95 | 1953.1 | 12.2 |
| Compound | −1.56 ± | 1500.55 ± | 6225.7 ± | 97.8 ± |
| no. 50, | 0.46 | 38.95 | 1502.6 | 9.5 |
| (6 mg/kg, | | | | |
| i.p. OD) | | | | |

TABLE 8
Effect of compound no. 7 on various metabolic parameters on repeated administration in DIO mice

| | % Change | Oxygen | AUC_glucose | AUC_Insulin | | Fasting | Rando [symbol] |
|---|---|---|---|---|---|---|---|
| Treatment | in body wt | consumption, | % change | % change | | plasma | plasm [symbol] |
| (dose) | from basal | ml/kg/h | during OGTT | during OGTT | HOMA-IR | TG (mg/dl) | TG,(mg/dl [symbol] |
| Vehicle control | −0.98 ± 1.31 | 2942.0 ± 66.3 | 5088.4 ± 788.8 | 4204.6 ± 1069.1 | 7.4 ± 1.47 | 44.5 ± 4.8 | 149.0 ± 17.4 [symbol] |
| Compound no. 7 | −3.19 ± 1.32 | 3116.3 ± 106.1 | 4526.7 ± 876.8 | 5431.0 ± 920.9 | 4.7 ± 1.68 | 40.4 ± 2.3 | 106.9 ± 13.7 [symbol] |
| (1.5 mg/kg, od) | | | | | | | |

[symbol]

indicates data missing or illegible when filed

TABLE 9
Effect of compound no. 50 on various metabolic parameters on
repeated administration in DIO mice

| | % | | AUC_ | AUC_ | |
|---|---|---|---|---|---|
| | Change | Oxygen | glucose | Insulin | |
| | in body | consump- | % change | % change | |
| Treatment | wt from | tion, | during | during | HOMA- |
| (dose) | basal | ml/kg/h | OGTT | OGTT | IR |
| Vehicle | −1.16 ± | 2561.0 2± | 8484.9 ± | 10604.4 ± | 3.48 ± |
| control | 1.29 | 38.9 | 1218.3 | 2555.3 | 1.07 |
| Compound | −3.29 ± | 2767.50 ± | 7763.5 ± | 12938.8 ± | 1.96 ± |
| no. 50 | 1.02 | 98.4 | 1251.2 | 1923.5 | 0.45 |
| (9 mg/kg, | | | | | |
| od) | | | | | |

1. N-(4-chlorophenyl)-2-(4-fluorophenoxy)-N-methylimidazo[1,2-a]pyrimidine-3-carboxamide or pharmaceutically acceptable salts thereof.

2. A pharmaceutical composition comprising a therapeutically effective amount of a compound as claimed in claim 1, in association with a pharmaceutically acceptable adjuvant, diluent or carrier.

3. A method of treating cardiometabolic disorders including diabetes, obesity, dyslipidemia, metabolic syndrome, atherosclerosis and non alcoholic fatty liver disease by administering a therapeutically effective amount of a compound as claimed in claim 1 to a mammal in need thereof.

4. Use of a compound as claimed in claim 1 for the preparation of a medicament for treating cardiometabolic disorders including diabetes, obesity, dyslipidemia, metabolic syndrome, atherosclerosis and non alcoholic fatty liver disease.

5. A compound of formula (I), 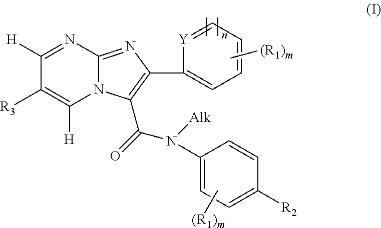
their pharmaceutically acceptable salts and their isomers, stereoisomers, conformers, tautomers, polymorphs, hydrates and solvates;
wherein,
n=0 or 1;
m=0, 1, 2or 3;
Y═CH, N or S, wherein ring containing Y group, may be optionally attached to imidazole ring through —NH, —N(alkyl), O or S;
Alk is optionally substituted straight chain alkyl, branched chain alkyl or cycloalkyl;
R₁ is selected from the group consisting of hydrogen, halo, cyano, nitro, C₁₋₈alkyl, hydroxy, —O—C₁₋₈alkyl, —CF₃, —N(R₄)(CO-alkyl), —N(R₄)(SO₂-aryl), —N(R₄)(SO₂-heteroaryl), —N(R₄)(SO₂-heterocyclyl), —N(R₄)(C(O)O—R₄), —N(R₄)(C(O)O-aryl), —N(R₄)(C(O)O-heteroaryl), —N(R₄)(C(O)O-heterocyclyl), —N(R₄)C(O)N(R₄)(R₄), —N(R₄)C(O)N(R₄)(aryl), —N(R₄)C(O)N(R₄)(heteroaryl), —N(R₄)C(O)N(R₄)(heterocyclyl), —N(R₄)SO₂N(R₄)(R₄), —N(R₄)SO₂N(R₄)(aryl), —N(R₄)SO₂N(R₄)(heteroaryl), —N(R₄)SO₂N(R₄)(heterocyclyl), —OC(O)(R₄), —O(aryl), —O(heteroaryl), —O(heterocyclyl), —S(R₄), —S-aryl, —S-heteroaryl, —S-heterocyclyl, —N(R₄)(R₄), —N(R₄)(aryl), —N(R₄)(heteroaryl), —N(R₄)(heterocyclyl), —C(O)(R₄), —C(O)(aryl), —C(O)(heteroaryl), —C(O)(heterocyclyl), —C(O)N(R₄)(R₄), —C(O)N(R₄)(aryl), —C(O)N(R₄)(heteroaryl), —C(O)N(R₄)(heterocyclyl), —C(O)O—(R₄), —C(O)O-aryl, —C(O)O-heteroaryl, —C(O)O-heterocyclyl, —S(O)(aryl), —S(O)(heteroaryl), —S(O)(heterocyclyl), —SO₂(aryl), —SO₂(heteroaryl), —SO₂(heterocyclyl), —SO₂N(R₄)(R₄), —SO₂N(R₄)(aryl), —SO₂N(R₄)(heteroaryl), —SO₂N(R₄)(heterocyclyl), aryl, heteroaryl and heterocyclyl;
R₂ is selected from the group consisting of halo, cyano, nitro, C₁₋₈alkyl, hydroxy, CF₃, —OCF₃, -amino, —O(C₁₋₈alkyl), —O(aryl), —O(heteroaryl), —O(heterocyclyl), —S(R₄), —S-aryl, —S-heteroaryl, —S-heterocyclyl, —C(O)O—(R₄), —C(O)O-aryl, —C(O)O-heteroaryl, —C(O)O— heterocyclyl, —SO₂N(R₄)(R₄), —SO₂N(R₄)(aryl), —SO₂N(R₄)(heteroaryl), —SO₂N(R₄)(heterocyclyl), aryl, heteroaryl and heterocyclyl; or
R₁ & R₂ when present on adjacent carbon atom may join together to form cycloalkenyl, aryl, heteroaryl or heterocyclyl ring;
R₃ is selected from the group consisting of hydrogen, cyano, nitro, hydroxy, —O(C₁₋₈ alkyl), —OCF₃, —N(R₄)(CO—R₄), —N(R₄)(CO-aryl), —N(R₄)(CO-heteroaryl), —N(R₄)(SO₂—R₄), —N(R₄)(SO₂—CF₃), —N(R₄)(SO₂-aryl), —N(R₄)(SO₂-heteroaryl), —N(R₄)(S O₂-heterocyclyl), —N(R₄)(C(O)O—R₄), —N(R₄)(C(O)O-aryl), —N(R₄)(C(O)O-heteroaryl), —N(R₄)(C(O)O— heterocyclyl), —N(R₄)C(O)N(R₄)(R₄), —N(R₄)C(O)N(R₄)(aryl), —N(R₄)C(O)N(R₄)(heteroaryl), —N(R₄)C(O)N(R₄)(heterocyclyl), —O(aryl), —O(heteroaryl), —O(heterocyclyl), —S(R₄), —S-aryl, —S-heteroaryl, —S-heterocyclyl, —N(R₄)(R₄), —N(R₄)(aryl), —N(R₄)(heteroaryl), —N(R₄)(heterocyclyl), —C(O)(R₄), —C(O)(aryl), —C(O)(heteroaryl), —C(O)(heterocyclyl), —C(O)N(R₄)(R₄), —C(O)N(R₄)(aryl), —C(O)N(R₄)(heteroaryl), —C(O)N(R₄)(heterocyclyl), —C(O)O—(R₄), —C(O)O-aryl, —C(O)O-heteroaryl, —C(O)O-heterocyclyl, S(O)—(C₁₋₈alkyl), —S(O)(aryl), —S(O)(heteroaryl), —S(O)(heterocyclyl), —SO₂ (C₁₋₈alkyl), —SO₂(aryl), —SO₂(heteroaryl), —SO₂(heterocyclyl), —SO₂N(R₄)(R₄), —SO₂N(R₄)(aryl), —SO₂N(R₄)(heteroaryl), —SO₂N(R₄)(heterocyclyl) and SO₂N(R₄)(cycloalkyl); and
R₄ is hydrogen or —C₁₋₈alkyl.

6. The compound of formula (I) as claimed in claim 5, wherein,
R₁ is selected from the group consisting of hydrogen, halo, cyano, hydroxy, —O—C₁₋₈ alkyl, —CF₃, —OCF₃, —N(R₄)(SO₂-aryl), —N(R₄)(SO₂-heteroaryl), —N(R₄)(SO₂-heterocyclyl), —N(R₄)(C(O)O—R₄), —N(R₄)(C(O)O-aryl), —N(R₄)(C(O)O-heteroaryl), —N(R₄)(C(O)O— heterocyclyl), —N(R₄)C(O)N(R₄)(R₄), —N(R₄)SO₂N(R₄)(aryl), _—O(aryl), —S(R₄), —N(R₄)(R₄), —N(R₄)(aryl), —C(O)(heterocyclyl), —C(O)N(R₄)(R₄), —C(O)N(R₄)(aryl), —SO₂(aryl), —SO₂N(R₄)(R₄), —SO₂N(R₄)(aryl), aryl, heteroaryl and heterocyclyl;
R₂ is selected from the group consisting of halo, cyano, C₁₋₈alkyl, hydroxy, CF₃, —OCF₃, -amino, —O(C₁₋₈alkyl), —O(aryl), —S-aryl, —C(O)O—(R₄), —SO₂N(R₄)(R₄), —SO₂N(R₄)(aryl), aryl, heteroaryl and heterocyclyl; and
R₃ is selected from the group consisting of hydrogen, cyano, nitro, hydroxy, —O(C₁₋₈alkyl), —N(R₄)SO₂(aryl), —N(R₄)(C(O)O—R₄), —N(R₄)C(O)N(R₄)(R₄),—N(R₄)C(O)N(R₄)(aryl), —N(R₄)(R₄), —C(O)(heterocyclyl),—C(O)O—(R₄), —SO₂(aryl) and —SO₂N(R₄)(aryl).

7. The compound of formula (I) as claimed in claim 5, wherein,
m=0, 1 or 2;
Y═CH, N or S, wherein ring containing Y group, may be optionally attached to imidazole ring through —NH or O;
R₁ is selected from the group consisting of hydrogen, halo, cyano, C₁₋₈alkyl, hydroxy, —CF₃, —OCF₃, —N(R₄)(R₄), —SO₂N(R₄)(R₄), aryl, heteroaryl and heterocyclyl;
R₂ is selected from the group consisting of halo, C₁₋₈alkyl, hydroxy, —O(C₁₋₈alkyl), —C(O)O—(R₄), —SO₂N(R₄)(R₄) and heterocyclyl; or
R₁ & R₂ when present on adjacent carbon atom may join together to form cycloalkenyl, ring; and
R₃ is selected from the group consisting of hydrogen, hydroxy, —N(R₄)(C(O)O—R₄), N(R₄)C(O)N(R₄)(R₄), —N(R₄)(R₄), —C(O)(heterocyclyl), C(O)O—(R₄) and SO₂(aryl).

8. The compound as claimed in claim 5, which is selected from the group consisting of:
N-(4-chlorophenyl)-N-methyl-2-phenylimidazo[1,2-a]pyrimidine-3-carboxamide;
N-(3-chlorophenyl)-N-methyl-2-phenylimidazo[1,2-a]pyrimidine-3-carboxamide;
(4-{methyl[2-phenylimidazo[1,2-a]pyrimidin-3-yl)carbonyl]amino}phenyl) acetic acid;
2-(4-chlorophenyl)-N-(2,3-dihydro-1H-inden-5-yl)-N-methylimidazo[1,2-a]pyrimidine-3-carboxamide;
N,2-bis(4-chlorophenyl)-N-methylimidazo[1,2-a]pyrimidine-3-carboxamide;
N-(2,3-dihydro-1H-inden-5-yl)-2-(4-fluorophenyl)-N-methylimidazo[1,2-a]pyrimidine-3-carboxamide;
N-(4-chlorophenyl)-2-(4-fluorophenyl)-N-methylimidazo[1,2-a]pyrimidine-3-carboxamide
2-(biphenyl-4-yl)-N-(4-chlorophenyl)-N-methylimidazo[1,2-a]pyrimidine-3-carboxamide;
2-(biphenyl-4-yl)-N-(2,3-dihydro-1H-inden-5-yl)-N-methylimidazo[1,2-a]pyrimidine-3-carboxamide;
N-(4-chlorophenyl)-2-(3,4-dimethoxyphenyl)-N-methylimidazo[1,2-a]pyrimidine-3-carboxamide;
N-(4-chlorophenyl)-N-methyl-2-(pyridin-2-yl)imidazo[1,2-a]pyrimidine-3-carboxamide;
N-(4-chlorophenyl)-N-methyl-2,6-diphenylimidazo[1,2-a]pyrimidine-3-carboxamide;
N-(2,3-dihydro-1H-inden-5-yl)-2-(3,4-dimethoxyphenyl)-N-methylimidazo[ 1,2-a]pyrimidine-3-carboxamide;
N-(4-chlorophenyl)-2-(3,4-dichlorophenyl)-N-methylimidazo[1,2-a]pyrimidine-3-carboxamide;
N-(2,3-dihydro-1H-inden-5-yl)-2-(3,4-dihydroxyphenyl)-N-methylimidazo[1,2-a]pyrimidine-3-carboxamide;
N-(4-chlorophenyl)-2-(4-methoxyphenyl)-N-methylimidazo[1,2-a]pyrimidine-3-carboxamide;
N-(2,3-dihydro-1H-inden-5-yl)-N-methyl-2-(pyridin-2-yl)imidazo[1,2-a]pyrimidine-3-carboxamide;
2-(4-chlorophenyl)-N-(4-fluorophenyl)-N-methylimidazo[1,2-a]pyrimidine-3-carboxamide;
2-(4-chlorophenyl)-N-(3,4-dimethoxyphenyl)-N-methylimidazo[1,2-a]pyrimidine-3-carboxamide;
N-(4-chlorophenyl)-N-methyl-2-(pyridin-3-yl)imidazo[1,2-a]pyrimidine-3-carboxamide;
2-(2,4-dichlorophenyl)-N-(4-fluorophenyl)-N-methylimidazo[1,2-a]pyrimidine-3-carboxamide;
N,2-bis(4-fluorophenyl)-N-methylimidazo[1,2-a]pyrimidine-3-carboxamide;
N-(4-chlorophenyl)-N-methyl-2-[2-(trifluoromethyl)phenyl]imidazo[1,2-a]pyrimidine-3-carboxamide;
2-(4-chlorophenyl)-N-[4-(dimethylsulfamoyl)phenyl]-N-methylimidazo[1,2-a]pyrimidine-3-carboxamide;
2-(4-chlorophenyl)-N-(2,4-dimethoxyphenyl)-N-methylimidazo[1,2-a]pyrimidine-3-carboxamide;
N-(4-fluorophenyl)-N-methyl-2-phenylimidazo[1,2-a]pyrimidine-3-carboxamide;
2-(4-chlorophenyl)-N-(3,4-dihydroxyphenyl)-N-methylimidazo[1,2-a]pyrimidine-3-carboxamide;
2-(1,3-benzodioxol-5-yl)-N-(4-fluorophenyl)-N-methylimidazo[1,2-a]pyrimidine-3-carboxamide;
2-(1,3-benzodioxol-5-yl)-N-(4-chlorophenyl)-N-methylimidazo[1,2-a]pyrimidine-3-carboxamide;
N-(4-chlorophenyl)-2-(4-hydroxyphenyl)-N-methylimidazo[1,2-a]pyrimidine-3-carboxamide;
N-(4-chlorophenyl)-N-methyl-2-[3-(trifluoromethyl)phenyl]imidazo[1,2-a]pyrimidine-3-carboxamide;
N-(4-chlorophenyl)-2-(4-fluorophenyl)imidazo[1,2-a]pyrimidine-3-carboxamide;
N-(4-fluorophenyl)-N-methyl-2-[3-(trifluoromethyl)phenyl]imidazo[1,2-a]pyrimidine-3-carboxamide;
N-(2,4-dimethoxyphenyl)-2-(4-fluorophenyl)imidazo[1,2-a]pyrimidine-3-carboxamide;
2-(3-cyanophenyl)-N-(4-fluorophenyl)-N-methylimidazo[1,2-a]pyrimidine-3-carboxamide;
N-(4-chlorophenyl)-2-(3-cyanophenyl)-N-methylimidazo[1,2-a]pyrimidine-3-carboxamide;
N-(4-chlorophenyl)-2-[4-(dimethylsulfamoyl)phenyl]-N-methylimidazo[1,2-a]pyrimidine-3-carboxamide;
2-[4-(dimethylsulfamoyl)phenyl]-N-(4-fluorophenyl)-N-methylimidazo[1,2-a]pyrimidine-3-carboxamide;
N-(2,4-difluorophenyl)-2-(4-fluorophenyl)imidazo[1,2-a]pyrimidine-3-carboxamide;
N-(2,4-difluorophenyl)-2-(4-fluorophenyl)-N-methylimidazo[1,2-a]pyrimidine-3-carboxamide;
2-(3-fluoro-4-methoxyphenyl)-N-(4-fluorophenyl)-N-methylimidazo[1,2-a]pyrimidine-3-carboxamide;
2-(3-fluoro-4-hydroxyphenyl)-N-(4-fluorophenyl)-N-methylimidazo[1,2-a]pyrimidine-3-carboxamide;
N-(2,4-difluorophenyl)-2-(3-fluoro-4-methoxyphenyl)-N-methylimidazo[1,2-a]pyrimidine-3-carboxamide;
N-(4-chlorophenyl)-2-(3-fluoro-4-methoxyphenyl)-N-methylimidazo[1,2-a]pyrimidine-3-carboxamide;
N-(3-chloro-4-fluorophenyl)-2-(3-fluoro-4-methoxyphenyl)-N-methylimidazo[1,2-a]pyrimidine-3-carboxamide;
N-(2,4-difluorophenyl)-2-(4-fluorophenoxy)-N-methylimidazo[1,2-a]pyrimidine-3-carboxamide;
N-(4-chlorophenyl)-N-methyl-2-phenoxyimidazo[1,2-a]pyrimidine-3-carboxamide;
2-(4-chlorophenoxy)-N-(4-chlorophenyl)-N-methylimidazo[1,2-a]pyrimidine-3-carboxamide;
N-(4-chlorophenyl)-2-(4-methoxyphenoxy)-N-methylimidazo[1,2-a]pyrimidine-3-carboxamide;
2-(4-chlorophenoxy)-N-(4-fluorophenyl)-N-methylimidazo[1,2-a]pyrimidine-3-carboxamide;
2-(4-fluorophenoxy)-N-(4-methoxyphenyl)-N-methylimidazo[1,2-a]pyrimidine-3-carboxamide;
N-(4-chlorophenyl)-2-(3-fluoro-4-hydroxyphenyl)-N-methylimidazo[1,2-a]pyrimidine-3-carboxamide;
N-(4-chlorophenyl)-2-(4-cyanophenyl)-N-methylimidazo[1,2-a]pyrimidine-3-carboxamide;
2-(4-cyanophenyl)-N-(4-fluorophenyl)-N-methylimidazo[1,2-a]pyrimidine-3-carboxamide;
N-(3-chloro-4-fluorophenyl)-2-(4-cyanophenyl)-N-methylimidazo[1,2-a]pyrimidine-3-carboxamide;
N-(4-chlorophenyl)-N-(4-fluorobenzyl)-2-(4-fluorophenyl)imidazo[1,2-a]pyrimidine-3-carboxamide;
2-(2,6-dichlorophenoxy)-N-(2,4-difluorophenyl)-N-methylimidazo[1,2-a]pyrimidine-3-carboxamide;
2-(2,6-dichlorophenoxy)-N-(4-fluorophenyl)-N-methylimidazo[1,2-a]pyrimidine-3-carboxamide;
N-(4-chlorophenyl)-2-(2,4-difluorophenoxy)-N-methylimidazo[1,2-a]pyrimidine-3-carboxamide;
2-(5-chlorothiophen-2-yl)-N-(4-fluorophenyl)-N-methylimidazo[1,2-a]pyrimidine-3-carboxamide;
N-(4-chlorophenyl)-2-(5-chlorothiophen-2-yl)-N-methylimidazo[1,2-a]pyrimidine-3-carboxamide;
N-(4-chlorophenyl)-N-methyl-2-[4-(trifluoromethyl)phenyl]imidazo[1,2-a]pyrimidine-3-carboxamide;
N-(4-fluorophenyl)-N-methyl-2-[4-(trifluoromethyl)phenyl]imidazo[1,2-a]pyrimidine-3-carboxamide;
N-(4-chlorophenyl)-2-(4-fluorophenyl)-N-(propan-2-yl)imidazo[1,2-a]pyrimidine-3-carboxamide;
N-(4-chlorophenyl)-2-(4-fluorophenoxyl)imidazo[1,2-a]pyrimidine-3-carboxamide;
N-(4-fluorophenyl)-2-[3-(trifluoromethyl)phenyl]imidazo[1,2-a]pyrimidine-3-carboxamide;
N-cyclopropyl-N,2-bis(4-fluorophenyl)imidazo[1,2-a]pyrimidine-3-carboxamide;
N-cyclopropyl-N-(4-fluorophenyl)-2-[3-(trifluoromethyl)phenyl]imidazo[1,2-a]pyrimidine-3-carboxamide;
N-(4-chlorophenyl)-N-cyclopropyl-2-[3-(trifluoromethyl)phenyl]imidazo[1,2-a]pyrimidine-3-carboxamide;
methyl {2-(4-fluorophenyl)-3-[(4-fluorophenyl)(methyl)carbamoyl]imidazo[1,2-a]pyrimidin-6-yl}carbamate;
methyl {3-[(4-chlorophenyl)(methyl)carbamoyl]-2-(4-fluorophenyl)imidazo[1,2-a]pyrimidin-6-yl}carbamate;
6-(carbamoylamino)-N-(4-chlorophenyl)-2-(4-fluorophenyl)-N-methylimidazo[1,2-a]pyrimidine-3-carboxamide;
6-(carbamoylamino)-N,2-bis(4-fluorophenyl)-N-methylimidazo[1,2-a]pyrimidine-3-carboxamide;
6-(carbamoylamino)-2-(4-fluorophenyl)-N-(4-methoxyphenyl)-N-methylimidazo[1,2-a]pyrimidine-3-carboxamide;
N-(4-chlorophenyl)-2-[3-(dimethylamino)phenyl]-N-methylimidazo[1,2-a]pyrimidine-3-carboxamide;
2-[3-(dimethylamino)phenyl]-N-(4-fluorophenyl)-N-methylimidazo[1,2-a]pyrimidine-3-carboxamide;
N-(4-chlorophenyl)-2-(5-fluoro-2-hydroxyphenyl)-N-methylimidazo[1,2-a]pyrimidine-3-carboxamide;
2-(5-fluoro-2-hydroxyphenyl)-N-(4-fluorophenyl)-N-methylimidazo[1,2-a]pyrimidine-3-carboxamide;
2-(5-fluoro-2-hydroxyphenyl)-N-(4-methoxyphenyl)-N-methylimidazo[1,2-a]pyrimidine-3-carboxamide;
N-(4-chlorophenyl)-N-methyl-2-[4-(propan-2-yl)phenyl]imidazo[1,2-a]pyrimidine-3-carboxamide;
N-(4-fluorophenyl)-N-methyl-2-[4-(propan-2-yl)phenyl]imidazo[1,2-a]pyrimidine-3-carboxamide;
N-(4-chlorophenyl)-2-(2,6-dihydroxyphenyl)-N-methylimidazo[1,2-a]pyrimidine-3-carboxamide;
2-(2,6-dihydroxyphenyl)-N-(4-fluorophenyl)-N-methylimidazo[1,2-a]pyrimidine-3-carboxamide;
N-(4-chlorophenyl)-2-(2-hydroxy-6-methoxyphenyl)-N-methylimidazo[1,2-a]pyrimidine-3-carboxamide;
N-(4-fluorophenyl)-2-(2-hydroxy-6-methoxyphenyl)-N-methylimidazo[1,2-a]pyrimidine-3-carboxamide;
N-(4-chlorophenyl)-N-methyl-2-(thiophen-2-yl)imidazo[1,2-a]pyrimidine-3-carboxamide;
N-(4-fluorophenyl)-N-methyl-2-(thiophen-2-yl)imidazo[1,2-a]pyrimidine-3-carboxamide;
N-(4-chlorophenyl)-2-(5-fluoro-2-hydroxyphenyl)-N-methylimidazo[1,2-a]pyrimidine-3-carboxamide;
2-(5-fluoro-2-hydroxyphenyl)-N-(4-fluorophenyl)-N-methylimidazo[1,2-a]pyrimidine-3-carboxamide;
N-(4-chlorophenyl)-2-(furan-2-yl)-N-methylimidazo[1,2-a]pyrimidine-3-carboxamide;
N-(4-fluorophenyl)-2-(furan-2-yl)-N-methylimidazo[1,2-a]pyrimidine-3-carboxamide;
N-(4-chlorophenyl)-2-(2,3-dihydro-1,4-benzodioxin-5-yl)-N-methylimidazo[1,2-a]pyrimidine-3-carboxamide;
2-(2,3-dihydro-1,4-benzodioxin-5-yl)-N-(4-fluorophenyl)-N-methylimidazo[1,2-a]pyrimidine-3-carboxamide;
N-(4-chlorophenyl)-2-(2-fluoro-4-methoxyphenyl)-N-methylimidazo[1,2-a]pyrimidine-3-carboxamide;
2-(2-fluoro-4-methoxyphenyl)-N-(4-fluorophenyl)-N-methylimidazo[1,2-a]pyrimidine-3-carboxamide;
N-(4-chlorophenyl)-N-methyl-2-[3-(trifluoromethoxy)phenyl]imidazo[1,2-a]pyrimidine-3-carboxamide;
N-(4-fluorophenyl)-N-methyl-2-[3-(trifluoromethoxy)phenyl]imidazo[1,2-a]pyrimidine-3-carboxamide;
N-(4-chlorophenyl)-N-methyl-2-[4-(methylsulfonyl)phenyl]imidazo[1,2-a]pyrimidine-3-carboxamide;
N-(4-fluorophenyl)-N-methyl-2-[4-(methylsulfonyl)phenyl]imidazo[1,2-a]pyrimidine-3-carboxamide;
N-(4-chlorophenyl)-2-[(4-methoxyphenyl)amino]-N-methylimidazo[1,2-a]pyrimidine-3-carboxamide;
N-(4-fluorophenyl)-2-[(4-methoxyphenyl)amino]-N-methylimidazo[1,2-a]pyrimidine-3-carboxamide;
N-(4-chlorophenyl)-2-[(4-chlorophenyl)amino]-N-methylimidazo[1,2-a]pyrimidine-3-carboxamide;
2-[(4-chlorophenyl)amino]-N-(4-fluorophenyl)-N-methylimidazo[1,2-a]pyrimidine-3-carboxamide;
N-(4-chlorophenyl)-2-[(4-fluorophenyl)amino]-N-methylimidazo[1,2-a]pyrimidine-3-carboxamide;
N-(4-fluorophenyl)-2-[(4-fluorophenyl)amino]-N-methylimidazo[1,2-a]pyrimidine-3-carboxamide;
N-(4-chlorophenyl)-2-[(4-methoxyphenyl)amino]-N-methylimidazo[1,2-a]pyrimidine-3-carboxamide;
N-(4-fluorophenyl)-2-[(4-methoxyphenyl)amino]-N-methylimidazo[1,2-a]pyrimidine-3-carboxamide;
N-(4-chlorophenyl)-2-[(3-fluoro-4-hydroxyphenyl)amino]-N-methylimidazo[1,2-a]pyrimidine-3-carboxamide;
N-(4-chlorophenyl)-N-methyl-2-{[2-(trifluoromethyl)phenyl]amino}imidazo[1,2-a]pyrimidine-3-carboxamide;
N-(4-fluorophenyl)-N-methyl-2-{[2-(trifluoromethyl)phenyl]amino}imidazo[1,2-a]pyrimidine-3-carboxamide;
N-(4-chlorophenyl)-N-methyl-2-{[3-(trifluoromethyl)phenyl]amino}imidazo[1,2-a]pyrimidine-3-carboxamide;
N-(4-fluorophenyl)-N-methyl-2-{[3-(trifluoromethyl)phenyl]amino}imidazo[1,2-a]pyrimidine-3-carboxamide;
N-(4-chlorophenyl)-6-(dimethylamino)-2-(4-fluorophenyl)-N-methylimidazo[1,2-a]pyrimidine-3-carboxamide;
N-(4-chlorophenyl)-2-[(4-fluorophenyl)amino]-N-methylimidazo[1,2-a]pyrimidine-3-carboxamide;
methyl {3-[(4-chlorophenyl)(methyl)carbamoyl]-2-(4-fluorophenyl)imidazo[1,2-a]pyrimidin-6-yl}methylcarbamate;
N-(4-chlorophenyl)-2-(4-fluorophenyl)-5,7-dihydroxy-N-methylimidazo[1,2-a]pyrimidine-3-carboxamide;
N-(4-chlorophenyl)-2-(4-fluorophenyl)-N,7-dimethylimidazo[1,2-a]pyrimidine-3-carboxamide;
N-(4-chlorophenyl)-2-(4-fluorophenyl)-N-methyl-6-[methyl(propan-2-ylcarbamoyl)amino]imidazo[1,2-a]pyrimidine-3-carboxamide;
N-(4-chlorophenyl)-2-(4-fluorophenyl)-7-hydroxy-N-methylimidazo[1,2-a]pyrimidine-3-carboxamide;
N-(4-chlorophenyl)-2-(4-fluorophenyl)-7-hydroxyimidazo[1,2-a]pyrimidine-3-carboxamide;
N-(4-chlorophenyl)-2-(4-fluorophenyl)-5-hydroxy-N-methylimidazo[1,2-a]pyrimidine-3-carboxamide;
N-(4-chlorophenyl)-2-(4-fluorophenyl)-5-hydroxyimidazo[1,2-a]pyrimidine-3-carboxamide;
N-(4-chlorophenyl)-2-(4-fluorophenyl)-7-hydroxy-N-(hydroxymethyl)imidazo[1,2-a]pyrimidine-3-carboxamide;
N-(4-chlorophenyl)-2-(4-fluorophenyl)-N-(hydroxymethyl)imidazo[1,2-a]pyrimidine-3-carboxamide;
N-(4-chlorophenyl)-2-(4-fluoro-2-hydroxyphenyl)-N-methylimidazo[1,2-a]pyrimidine-3-carboxamide;
N-(4-chloro-2-hydroxyphenyl)-2-(4-fluorophenyl)-N-methylimidazo[1,2-a]pyrimidine-3-carboxamide;
3-[(4-chlorophenyl)(methyl)carbamoyl]-2-(4-fluorophenyl)imidazo[1,2-a]pyrimidine-6-carboxylic acid;
N-(4-chlorophenyl)-2-(4-fluorophenyl)-N-methyl-6-(phenylsulfonyl)imidazo[1,2-a]pyrimidine-3-carboxamide;
N-(4-chlorophenyl)-2-(4-fluorophenyl)-N-methyl-6-(pyrrolidin-1-ylcarbonyl)imidazo[1,2-a]pyrimidine-3-carboxamide;
N-(4-fluorophenyl)-N-methyl-2-(morpholin-4-yl)imidazo[1,2-a]pyrimidine-3-carboxamide;
N-(4-chlorophenyl)-2-(3-fluorophenoxy)-N-methylimidazo[1,2-a]pyrimidine-3-carboxamide;
2-(4-fluorophenyl)-N-(4-methoxyphenyl)-N-methylimidazo[1,2-a]pyrimidine-3-carboxamide;
ethyl 4-[{[2-(4-fluorophenyl)imidazo[1,2-a]pyrimidin-3-yl]carbonyl}(methyl)amino]benzoate;
N-(4-chlorophenyl)-2-(4-fluoro-2-methoxyphenyl)-N-methylimidazo[1,2-a]pyrimidine-3-carboxamide;
N-(4-chlorophenyl)-N-cyclopropyl-2-(4-fluorophenyl)imidazo[1,2-a]pyrimidine-3-carboxamide;
N-(4-chlorophenyl)-2-(4-fluorophenyl)-N-methylimidazo[1,2-a]pyrimidine-3-carboxamide hydrochloride (1:1);
N-(4-chlorophenyl)-2-(4-fluorophenyl)-N-methylimidazo[1,2-a]pyrimidine-3-carboxamide sulfate (1:1);
N-(4-chlorophenyl)-2-(4-fluorophenyl)-N-methylimidazo[1,2-a]pyrimidine-3-carboxamide 4-methylbenzenesulfonate (1:1);
N-(4-chlorophenyl)-2-(4-fluorophenyl)-N-methylimidazo[1,2-a]pyrimidine-3-carboxamide methanesulfonate (1:1);
2-(4-fluorophenyl)-N-methylimidazo[1,2-a]pyrimidine-3-carboxamide;
N-(4-cyclohexylphenyl)-2-(4-fluorophenyl)-N-methylimidazo[1,2-a]pyrimidine-3-carboxamide;
2-(4-fluorophenyl)-N-methyl-N-[4-(morpholin-4-yl)phenyl]imidazo[1,2-a]pyrimidine-3-carboxamide;
N-(biphenyl-3-yl)-2-(4-fluorophenyl)-N-methylimidazo[1,2-a]pyrimidine-3-carboxamide;
N-(4-chlorophenyl)-2-(4-fluoro-3-methoxyphenyl)-N-methylimidazo[1,2-a]pyrimidine-3-carboxamide;
N-(4-chlorophenyl)-N-methyl-2-{4-[(phenylsulfonyl)amino]phenyl}imidazo[1,2-a]pyrimidine-3-carboxamide;
methyl (4-{3-[(4-chlorophenyl)(methyl)carbamoyl]imidazo[1,2-a]pyrimidin-2-yl}phenyl)carbamate;
N-(4-chlorophenyl)-N-methyl-2-{4-[(methylcarbamoyl)amino]phenyl}imidazo[1,2-a]pyrimidine-3-carboxamide;
N-(4-chlorophenyl)-N-methyl-2-{4-[(phenylsulfamoyl)amino]phenyl}imidazo[1,2-a]pyrimidine-3-carboxamide;
N-(4-chlorophenyl)-2-[4-(4-fluorophenoxyl)phenyl]-N-methylimidazo[1,2-a]pyrimidine-3-carboxamide;
N-(4-chlorophenyl)-N-methyl-2-[4-(methylsulfanyl)phenyl]imidazo[1,2-a]pyrimidine-3-carboxamide;
N-(4-chlorophenyl)-2-{4-[(4-fluorophenyl)amino]phenyl}-N-methylimidazo[1,2-a]pyrimidine-3-carboxamide;
N-(4-chlorophenyl)-N-methyl-2-[4-(piperazin-1-ylcarbonyl)phenyl]imidazo[1,2-a]pyrimidine-3-carboxamide;
N-(4-chlorophenyl)-N-methyl-2-{4-[methyl(phenyl)carbamoyl]phenyl}imidazo[1,2-a]pyrimidine-3-carboxamide;
N-(4-chlorophenyl)-2-{4-[(4-fluorophenyl)sulfonyl]phenyl}-N-methylimidazo[1,2-a]pyrimidine-3-carboxamide;
N-(4-chlorophenyl)-2-{4-[(4-fluorophenyl)sulfamoyl]phenyl}-N-methyl imidazo[1,2-a]pyrimidine-3-carboxamide;
N-[4-(4-chlorophenoxy)phenyl]-2-(4-fluorophenyl)-N-methylimidazo[1,2-a]pyrimidine-3-carboxamide;
N-{4-[(4-chlorophenyl)sulfanyl]phenyl}-2-(4-fluorophenyl)-N-methylimidazo[1,2-a]pyrimidine-3-carboxamide;
N-methyl-2-phenyl-N-[4-(phenylsulfamoyl)phenyl]imidazo[1,2-a]pyrimidine-3-carboxamide;
N-(4-chlorophenyl)-2-(4-fluorophenyl)-N-methyl-6-[(phenylcarbonyl)amino]imidazo[1,2-a]pyrimidine-3-carboxamide;
N-(4-chlorophenyl)-2-(4-fluorophenyl)-N-methyl-6-[(phenylsulfonyl)amino]imidazo[1,2-a]pyrimidine-3-carboxamide;
N-(4-chlorophenyl)-2-(4-fluorophenyl)-N-methyl-6-[(phenylcarbamoyl)amino]imidazo[1,2-a]pyrimidine-3-carboxamide;
N-(4-chlorophenyl)-2-(4-fluorophenyl)-6-hydroxy-N-methylimidazo[1,2-a]pyrimidine-3-carboxamide; and
N-(4-chlorophenyl)-2-(4-fluorophenyl)-N-methyl-6-(phenylsulfamoyl) imidazo[1,2-a]pyrimidine-3-carboxamide or pharmaceutically acceptable salt thereof.
N-(4-chlorophenyl)-2-(4-fluorophenoxy)-N-methylimidazo[ 1,2-a]pyrimidine-3-carboxamide 4-methylbenzenesulfonate (1:1)
N-(4-chlorophenyl)-2-(4-fluorophenoxy)-N-methylimidazo[ 1,2-a]pyrimidine-3-carboxamide benzenesulfonate (1:1)
N-(4-chlorophenyl)-2-(4-fluorophenoxy)-6-hydroxy-N-methylimidazo[1,2-a]pyrimidine-3-carboxamide
N-(4-chlorophenyl)-2-(4-fluorophenoxy)-5-hydroxy-N-methylimidazo[1,2-a]pyrimidine-3-carboxamide
N-(4-chlorophenyl)-2-(4-fluorophenoxy)-7-hydroxy-N-methylimidazo[1,2-a]pyrimidine-3-carboxamide
N-(4-chlorophenyl)-2-(4-fluorophenoxy)-N-(hydroxymethyl)imidazo[1,2-a]pyrimidine-3-carboxamide
N-(4-chlorophenyl)-2-(4-fluoro-3-hydroxyphenoxy)-N-methylimidazo[1,2-a]pyrimidine-3-carboxamide
N-(4-chloro-3-hydroxyphenyl)-2-(4-fluorophenoxy)-N-methylimidazo[1,2-a]pyrimidine-3-carboxamide

9. The compound as claimed in claim 8, which is selected from the group consisting of:
N-(4-chlorophenyl)-N-methyl-2-phenylimidazo[1,2-a]pyrimidine-3-carboxamide;
N-(4-chlorophenyl)-2-(4-fluorophenyl)-N-methylimidazo[1,2-a]pyrimidine-3-carboxamide;
N-(4-chlorophenyl)-N-methyl-2-(pyridin-3-yl)imidazo[1,2-a]pyrimidine-3-carboxamide;
N,2-bis(4-fluorophenyl)-N-methylimidazo[1,2-a]pyrimidine-3-carboxamide;
N-(4-chlorophenyl)-2-(4-hydroxyphenyl)-N-methylimidazo[1,2-a]pyrimidine-3-carboxamide;
N-(4-fluorophenyl)-N-methyl-2-[3-(trifluoromethyl)phenyl]imidazo[1,2-a]pyrimidine-3-carboxamide;
N-(3-chloro-4-fluorophenyl)-2-(3-fluoro-4-methoxyphenyl)-N-methylimidazo[1,2-a]pyrimidine-3-carbox amide;
N-(4-chlorophenyl)-N-cyclopropyl-2-(4-fluorophenyl)imidazo[1,2-a]pyrimidine-3-carbox amide; and
N-(4-chlorophenyl)-2-(4-fluorophenyl)-N-(propan-2-yl)imidazo[1,2-a]pyrimidine-3-carboxamide or pharmaceutically acceptable salt thereof

10. A pharmaceutical composition comprising a therapeutically effective amount of one or more compound as claimed in claim 5, in association with a pharmaceutically acceptable adjuvant, diluent or carrier.

11. A method for treating cardiometabolic disorders including diabetes, obesity, dyslipidemia, metabolic syndrome, atherosclerosis and non alcoholic fatty liver disease by administering a therapeutically effective amount of a compound of formula (I) as claimed in claim 5 to a mammal in need thereof.

12. Use of a compound of formula (I) as claimed in claim 5 for the preparation of a medicament for treating cardiometabolic disorders including diabetes, obesity, dyslipidemia, metabolic syndrome, atherosclerosis and non alcoholic fatty liver disease.

13. Use of a compound of formula (I) as claimed in claim 5 for the preparation of salts, polymorphs, hydrates and solvates of compound of formula (I).

14. A process of preparation of polymorph of compound of formula (I) as claimed in claim 5, comprises contacting compound of formula (I) with suitable solvent or mixture of solvent

15. A compound of formula (I), its method for preparation and pharmaceutical composition, as herein described with reference to the examples accompanying the specification.
